(19) United States
(12) Patent Application Publication
(10) Pub. No.: US 2025/0120905 A1
(43) Pub. Date: Apr. 17, 2025

(54) THERAPEUTIC AGENTS TARGETING THE LYMPHATIC SYSTEM

(71) Applicant: VIVASOR, INC, San Diego, CA (US)

(72) Inventors: Russell F. Ross, San Diego, CA (US); Brian R. Cooley, San Diego, CA (US)

(21) Appl. No.: 18/684,580

(22) PCT Filed: Aug. 16, 2022
(86) PCT No.: PCT/US2022/040490

§ 371 Date: Feb. 16, 2024

Related U.S. Application Data
(60) Provisional application No. 63/316,123, filed on Mar. 3, 2022.
(60) Provisional application No. 63/234,683, filed on Aug. 18, 2021.

(51) Int. Cl. A61K 9/00; A61K 45/06; A61P 19/02

(52) U.S. Cl. CPC A61K 9/0021; A61K 45/06; A61P 19/02

(57) ABSTRACT

Disclosed herein, inter alia, are medical devices and methods for administering therapeutic agents to one or more positions comprising or proximal to one or more regions of the lymphatic system of a subject. Also disclosed herein are, inter alia, methods for treating a subject having, or suspected of having, an arthritic disease or associated condition, or a symptom of an arthritic disease of associated condition, by delivering a therapeutically effective amount of a therapeutic agent to the lymphatic system of the subject.

### CROSS REFERENCE TO RELATED APPLICATIONS

This application claims priority to U.S. provisional patent application No. 63/316,123, filed on Mar. 3, 2022, and U.S. provisional patent application No. 63/234,683, filed on Aug. 18, 2021, the contents and disclosures of which are incorporated by reference in their entireties for all purposes.

### FIELD OF THE INVENTION

The field of the disclosure relates generally to the administration of a therapeutic agent to the lymphatic system of a subject by use of a fluid delivery apparatus that enables the targeting of one or more components of the lymphatic system, such as initial lymphatic capillaries, collecting lymphatic vessels, afferent lymphatic vessels, and/or lymph nodes. In certain embodiments, this disclosure relates to devices and methods for treating a subject having, or suspected of having, an arthritic disease or associated condition, or one or more symptoms or clinical manifestations thereof, comprising administration of a therapeutically effective amount of a therapeutic agent to the lymphatic system of the subject.

### BACKGROUND OF THE INVENTION

The lymphatic system plays an important role in transporting body fluids and particulate materials throughout the body. The lymphatic system comprises several lymph organs (e.g., the spleen and thymus) in addition to lymph nodes, lymph vessels and lymph capillaries. The vessels transport lymph fluid around the body in a single direction in either the superficial vessels or the deep vessels (i.e., the lymphatic vasculature). Drainage begins in blind capillaries which gradually develop into vessels. These vessels then travel through several lymph nodes. The lymph nodes contain both T and B lymphocytes in addition to other cells associated with the immune system. Antigens and other foreign particles are filtered out in the lymph nodes. The lymph vessels eventually end in either the right lymphatic duct which drains into the right internal jugular vein or the thoracic duct which drains into the subclavian vein. It is a one-way system where the lymph fluid (also referred to a lymph) is eventually returned to the circulatory system of the patient.

Large proteins and certain cells (lymphocytes) pass from the blood plasma into the tissue fluid, and it is an important function of the lymph (i.e., the fluid in the lymphatic system) to return these essential components to the blood circulation. The lymph also plays an important role in transporting the products of fat digestion in the gastrointestinal tract, the chylomicrons, and into the blood circulation.

Numerous devices have been developed for transdermal drug delivery using microneedle assemblies or arrays. Microneedle assemblies reduce the amount of pain felt by a patient as compared to larger conventional needles. Moreover, conventional subcutaneous (and often intra-muscular) delivery of medicines using a needle operates to deliver a large quantity of the medicine at one time, thereby creating a spike in the bioavailability of the therapeutic agent. While this is not a significant problem for some medicaments, many medical conditions benefit from having a steady state concentration of the active therapeutic agent for an extended period of time. Transdermal delivery apparatus is capable of administering medicaments at a substantially constant rate over an extended period of time. Some devices are capable of delivering a medicament directly into the lymphatic system of a patient. Exemplary such devices comprise, available from Sorrento Therapeutics, Inc., are disclosed, for example, in WO 2011/135530; WO2011/135533; WO/2012/020332; WO 2014/132239; WO 2014/188343; WO 2015/168210; WO 2015/168215; WO 2015/168217; WO 2015/168219; WO2017/0189258; WO 2017/0189259; WO 2017/019535; WO 2018/111611; WO 2018/111616; and WO 2018/111621; the disclosures of which are hereby incorporated by reference in their entireties.

Rheumatoid arthritis (RA) is the most common autoimmune inflammatory arthritis affecting more than 1.3 million adults in the United States.¹ The overall worldwide prevalence varies between 0.3% and 1% and is more common in women and in people living in developed countries (WHO 2019). RA is a chronic disease primarily affecting the joints, connective tissues, muscle, tendons, and fibrous tissues. A patient with RA typically presents with pain and swelling in the joints of the hands and feet, resulting in significant impairment to their physical function and quality of life as the disease progresses. Additionally, it is not uncommon for patients with RA to develop other autoimmune conditions. About 25 percent of patients with one such condition tend to develop others (see, e.g., Cojocaru, M., Cojocaru, I. M. & Silosi, I. Multiple autoimmune syndrome. Maedica (Buchar). 5, 132-134 (2010)).

Although there is currently no cure for RA, the availability of new biologic treatments that directly target components of the RA inflammatory cascade has transformed management of the disease. Monoclonal antibodies and fusion proteins have been developed to modify the immune response that leads to inflammation and joint damage. A primary focus for current and emerging biologics for the treatment of RA is the immune system where adaptive immune responses are mounted. Because these therapeutics are administered intravenously (IV) or subcutaneously (SC), drug exposure to targets in the lymph nodes where immune modulation and activation take place may be limited. This can contribute to reduced efficacy or even non-responsiveness at high doses.

According to the American College of Rheumatology (ACR), the ultimate goals of RA treatment are the prevention or control of joint damage, the prevention of functional loss, and the relief of pain (ACR 2002). The clinical goal of RA treatment is to achieve disease control by optimizing a treat-to-target strategy aimed at reducing disease activity by at least 50% within 3 months and achieving remission or low disease activity (LDA) within 6 months (see, e.g., Aletaha, D., Alasti, F. & Smolen, J. S. Optimisation of a treat-to-target approach in rheumatoid arthritis: strategies for the 3-month time point. Ann. Rheum. Dis. 75, 1479-1485 (2016)). Clinical remission is a state in which physical function is maximally improved and progression of joint damage is slowed if not halted.

The ACR and the European League Against Rheumatism (EULAR) treatment guidelines recommend treatment of RA patients with disease-modifying antirheumatic drugs (DMARDs) (see, e.g, Singh, J. A. et al. 2015 American College of Rheumatology Guideline for the Treatment of Rheumatoid Arthritis. Arthritis Care Res. (Hoboken). 68, 1-25 (2016) and Smolen, J. S. et al. EULAR recommendations for the management of rheumatoid arthritis with synthetic and biological disease-modifying antirheumatic drugs: 2019 update. Ann. Rheum. Dis. 79, 685-699 (2020)). Methotrexate (MTX) is the first line DMARD used for treatment and is generally prescribed at an optimal dose up to 25 mg with or without glucocorticoids. If this treatment fails, sequential application of targeted therapies, such as biologic agents (e.g., tumor necrosis factor (TNF) inhibitors or Janus kinase (JAK)-inhibitors in combination with MTX are then recommended.

In particular, tumor necrosis factor alpha (TNF-α) has become a significant therapeutic target in connection with a large variety of medical conditions, including rheumatoid arthritis (RA), juvenile arthritis, psoriatic arthritis, plaque psoriasis, ankylosing spondylitis, ulcerative colitis (UC), and Crohn's disease. Multiple drugs that specifically target TNF-α have received FDA approval including Adalimumab (Humira®), Adalimumab-atto (Amjevita®, a biosimilar to Humira®), Certolizumab pegol (Cimzia®), etanercept (Enbrel®), etanercept-szzs (Ereizi®, a biosimilar to Enbrel®), Golimumab (Simponi®, Simponi Aria®), Infliximab (Remicade®), and Infliximab-dyyb (Inflectra®, a biosimilar to Remicade®), while literally dozens of clinical trials are ongoing with either new therapeutic agents or expanded uses for currently approved ones.

Among these, Enbrel® (etanercept subcutaneous injection), initially FDA approved in the US in 1998 for the treatment of RA, is a TNF inhibitor that is indicated for reducing signs and symptoms, inducing major clinical response, inhibiting the progression of structural damage, and improving physical function in patients with moderately to severely active RA. Enbrel is a fusion protein consisting of a portion of the TNF receptor linked to the fragment crystallizable (Fc) portion of human immunoglobulin G (IgG). By binding specifically to TNF, Enbrel blocks the interaction of TNF with cell surface TNF receptors, and thereby modulates the cellular immune response triggered when TNF binds to receptors on immune cells. Given that TNF receptors are present primarily (but not exclusively) on immune cells, the leukocytes concentrated in lymph nodes represent the primary reservoir of leukocyte-expressed TNF receptors. Enbrel is also indicated for treatment of Juvenile Idiopathic Arthritis (JIA), Psoriatic Arthritis (PsA), Ankylosing Spondylitis (AS) and Plaque Psoriasis (PsA).

While TNF inhibitor therapies delivered systemically such as Enbrel have shown rapid and sustained reductions in disease activity and are considered successful in the treatment of RA, some limitations exist in clinical response. The efficacy response rate of Enbrel in combination with MTX is limited (<50%) based on the composite measure of American College of Rheumatology (ACR) score of 70% improvement criteria (ACR70). ACR70 response rates correspond reasonably well with an overall low disease activity state (including remission). ACR70 requires at least a 70% improvement in tender joint count (TJC), swollen joint count (SJC), and at least a 70% improvement in 3 of the following 5 measures: Patient Assessment of Pain, Patient Global Assessment of Disease Activity, Physician Global Assessment of Disease Activity, Patient assessment of physical function using the Health Assessment Questionnaire-Disability Index (HAQ-DI), and Acute phase reactant: either C-reactive protein (CRP) or erythrocyte sedimentation rate (ESR).

Additionally, all RA drugs exhibit decreasing efficacy with increasing disease duration or drug exposure, even if they target a different biologic pathway than prior therapies. In MTX-naive patients with high disease activity, ACR70 response rates for treatment with biologic DMARDs (e.g., Enbrel) plus MTX are approximately 35% to 40%. In MTX-insufficient responders, this rate is about 20%, and in TNF inhibitor-insufficient responders, the rate is 10% to 15%. In addition, as reported in the Enbrel prescribing information, treatment with Enbrel plus MTX achieved ACR70 in only 15% of patients at 3 and 6 months (see, e.g., Enbrel USPI (2020)). Thus, there remains significant need for new therapies and treatment methods to improve response rates and treatment outcomes.

Furthermore, known side effects for TNF-α inhibitors include headaches, heartburn, nausea, vomiting, allergic reactions and muscle weakness. Because TNF-α plays an important role in the immune system, altering TNF-α activity makes a patient more susceptible to secondary infections or some cancers.

Accordingly, there is need to develop administration devices, methods, dosing regimens and the like that provide efficacious delivery of therapeutically effective amounts of therapeutic agents for treating arthritic diseases or associated conditions in subjects having, or suspected of having, one or more of arthritic diseases or associated conditions. There is also a need to provide administration devices and methods for delivering dose-sparing amounts or concentrations of such therapeutic agent so that, for example, the overall patient exposure to the therapeutic agent and associated and untoward off-target effects and/or side effects are reduced.

### SUMMARY OF THE INVENTION

In certain embodiments are provided methods of treating a subject having, or suspected of having, an arthritic disease or associated condition, or a symptom associated with an arthritic disease or associated condition, by administering a therapeutically effective amount of a therapeutic agent to the lymphatic system of, the method comprising: placing a first medical device comprising a plurality of microneedles on the skin of the subject at a first location proximate to a first position under the skin of the subject, wherein the first position is proximate to lymph vessels and/or lymph capillaries that drain into a lymphatic duct, and wherein the microneedles of the first medical device have a surface comprising nanotopography; inserting the plurality of microneedles of the first medical device into the subject to a depth whereby at least the epidermis is penetrated and an end of at least one of the microneedles is proximate to the first position; and administered via the microneedles of the first medical device a first dose of the anti-inflammatory agent into the first position; thereby delivering the therapeutically effective amount of the therapeutic agent to the lymphatic system of the subject.

In certain embodiments, which may be combined with other embodiments disclosed throughout, are provided methods of increasing lymphatic amount or lymphatic concentration of a therapeutic agent in the lymphatic system of a subject having, or suspected of having, an arthritic disease or associated condition, or a symptom associated with an arthritic disease or associated condition, the method comprising: placing a first medical device comprising a plurality of microneedles on the skin of the subject at a first location proximate to a first position under the skin of the subject, wherein the first position is proximate to lymph vessels and/or lymph capillaries that drain into a lymphatic duct, and wherein the microneedles of the first medical device have a surface comprising nanotopography; inserting the plurality of microneedles of the first medical device into the subject to a depth whereby at least the epidermis is penetrated and an end of at least one of the microneedles is proximate to the first position; and administered via the microneedles of the first medical device a first dose of the anti-inflammatory agent into the first position; thereby increasing the lymphatic concentration of the therapeutic agent in the lymphatic system of the subject.

In certain embodiments, which may be combined with other embodiments disclosed throughout, are provided methods of decreasing an elevated lymphatic amount or lymphatic concentration of an inflammatory substance in a subject having, or suspected of having, an arthritic disease or associated condition, or a symptom associated with an arthritic disease or associated condition, wherein the elevated lymphatic amount or lymphatic concentration results from the presence of the disease or associated condition, the method comprising: placing a first medical device comprising a plurality of microneedles on the skin of the subject at a first location proximate to a first position under the skin of the subject, wherein the first position is proximate to lymph vessels and/or lymph capillaries that drain into a lymphatic duct, and wherein the microneedles of the first medical device have a surface comprising nanotopography; inserting the plurality of microneedles of the first medical device into the subject to a depth whereby at least the epidermis is penetrated and an end of at least one of the microneedles is proximate to the first position; and administering via the microneedles of the first medical device a therapeutically effective amount of the therapeutic agent into the first position; wherein the therapeutically effective amount comprises an amount that results in a reduction in the lymphatic amount or lymphatic concentration of the inflammatory substance in the lymphatic system of the subject.

In certain embodiments, which may be combined with other embodiments disclosed throughout, are provided methods of increasing lymphatic pumping rate of at least on lymph node in a subject having, or suspected of having, an arthritic disease or associated condition, or a symptom associated with an arthritic disease or associated condition, the method comprising: placing a first medical device comprising a plurality of microneedles on the skin of the subject at a first location proximate to a first position under the skin of the subject, wherein the first position is proximate to lymph vessels and/or lymph capillaries that drain into a lymphatic duct, and wherein the microneedles of the first medical device have a surface comprising nanotopography; inserting the plurality of microneedles of the first medical device into the subject to a depth whereby at least the epidermis is penetrated and an end of at least one of the microneedles is proximate to the first position; and administering via the microneedles of the first medical device a therapeutically effective amount of the therapeutic agent into the first position; wherein the therapeutically effective amount comprises an amount that results in an increased lymphatic pumping rate of the at least on lymph node in the lymphatic system of the subject.

In certain embodiments, which may be combined with other embodiments disclosed throughout, are provided methods of achieving or restoring a normal pumping rate of at least one lymph node in a subject having, or suspected of having, an arthritic disease or associated condition, or a symptom associated with an arthritic disease or associated condition, wherein the pumping rate is reduced as a result of the arthritic disease or associated condition, the method comprising: placing a first medical device comprising a plurality of microneedles on the skin of the subject at a first location proximate to a first position under the skin of the subject, wherein the first position is proximate to lymph vessels and/or lymph capillaries that drain into a lymphatic duct, and wherein the microneedles of the first medical device have a surface comprising nanotopography; inserting the plurality of microneedles of the first medical device into the subject to a depth whereby at least the epidermis is penetrated and an end of at least one of the microneedles is proximate to the first position; and administering via the microneedles of the first medical device a therapeutically effective amount of the therapeutic agent into the first position; wherein the therapeutically effective amount comprises an amount that results in restoration of the normal pumping rate to a rate that is comparable to or greater than the pumping rate in a subject that does not have the disease or associated condition.

In certain embodiments, which may be combined with other embodiments disclosed throughout, a therapeutically effective in accordance with the disclosed methods amount comprises: an amount or concentration that is effective to treat the disease or associated condition; or an amount or concentration that is effective to reduce or eliminate at least one symptom or clinical manifestation of the disease or associated condition.

In certain embodiments, which may be combined with other embodiments disclosed throughout, an arthritic disease or associated condition that may be treated by one or more methods disclosed throughout is selected from the group consisting of: rheumatoid arthritis (RA); juvenile arthritis; psoriatic arthritis; ankylosing spondylitis; gout; and combinations thereof.

In certain embodiments, which may be combined with other embodiments disclosed throughout, an associated condition that may be treated by one or more methods disclosed throughout is another autoimmune condition. In certain embodiments, which may be combined with other embodiments disclosed throughout, the associated condition comprises an autoimmune condition selected from the group consisting of scleroderma, lupus ulcerative colitis (UC), Crohn's disease, plaque psoriasis, autoimmune uveitis, Behçet's disease, and sarcoidosis.

In certain embodiments, which may be combined with other embodiments disclosed throughout, a therapeutic agent comprises an anti-inflammatory agent. In certain embodiments, which may be combined with other embodiments disclosed throughout, the therapeutic agent comprises an anti-arthritic agent. In certain embodiments, which may be combined with other embodiments disclosed throughout, the therapeutic agent comprises an agent that reduces TNFa activity. In certain embodiments, which may be combined with other embodiments disclosed throughout, the therapeutic agent is selected from the group consisting of: Adalimumab (Humira®); Adalimumab-atto (Amjevita®); Certolizumab pegol (Cimzia®); etanercept (Enbrel®); etanercept-szzs (Ereizi®); Golimumab (Simponi®, Simponi Aria®); Infliximab (Remicade®); Infliximab-dyyb (Inflectra®); analogs thereof; variants thereof; biosimilars thereof; bioequivalents thereof; and combinations thereof.

In certain embodiments, which may be combined with other embodiments disclosed throughout, a therapeutically effective amount of a therapeutic agent administered according to any of the disclosed methods comprises a dose-sparing amount of the therapeutic agent.

In certain embodiments, which may be combined with other embodiments disclosed throughout, a therapeutically effective amount of a therapeutic agent administered according to any of the disclosed methods is effective to reduce a DAS28 (ESR) and/or a DAS28(CRT) score by at least 10%, at least 20%, at least 30%, at least 40%, at least 50%, at least 60%, at least 70%, or greater compared to a DAS28 (ESR) and/or a DAS28(CRT) determined in the subject prior to administering the therapeutic agent.

In certain embodiments, which may be combined with other embodiments disclosed throughout, a therapeutically effective amount of a therapeutic agent administered according to any of the disclosed methods is effective to reduce a 66/68-joint count and/or a 28-joint count score by at least 10%, at least 20%, at least 30%, at least 40%, at least 50%, at least 60%, at least 70%, or greater compared to a 66/68-joint count and/or a 28-joint count determined in the subject prior to administering the therapeutic agent.

In certain embodiments, which may be combined with other embodiments disclosed throughout, a therapeutically effective amount of a therapeutic agent administered according to any of the disclosed methods is effective to reduce patient rating of overall disease activity by at least 10%, at least 20%, at least 30%, at least 40%, at least 50%, at least 60%, at least 70%, or greater compared to a patient rating of overall disease activity determined in the subject prior to administering the therapeutic agent.

In certain embodiments, which may be combined with other embodiments disclosed throughout, a therapeutically effective amount of a therapeutic agent administered according to any of the disclosed methods is effective to improve ACR response by at least 10%, at least 20%, at least 30%, at least 40%, at least 50%, at least 60%, at least 70%, or greater compared to a an ACR response determined in the subject prior to administering the therapeutic agent.

In certain embodiments, which may be combined with other embodiments disclosed throughout, a subject treated according to any of the disclosed methods subject is a mammal.

In certain embodiments, which may be combined with other embodiments disclosed throughout, the subject is a human.

In certain embodiments, which may be combined with other embodiments disclosed throughout, a medical device for administering a therapeutically effective amount of a therapeutic agent to the lymphatic system of a subject comprises a Sofusa™ Lymphatic Delivery System (SOFUSA).

In certain embodiments, which may be combined with other embodiments disclosed throughout, a medical device for administering a therapeutically effective amount of a therapeutic agent to the lymphatic system of a subject comprises a fluid delivery apparatus, wherein the fluid delivery apparatus comprises: a fluid distribution assembly wherein a cap assembly is coupled to a cartridge assembly, and the cartridge assembly is slidably coupled to a plenum assembly, and a mechanical controller assembly is slidably coupled to the cartridge assembly; a collet assembly constituting the housing of the fluid delivery apparatus and being slidably coupled to the fluid distribution assembly; and a plurality of microneedles fluidically connected with the fluid distribution assembly having a surface comprising nanotopography, the plurality of microneedles being capable of penetrating the stratum corneum of the skin of a subject and controllably delivering the therapeutic agent to a depth below the surface of the skin of the subject. In certain embodiments, which may be combined with other embodiments disclosed throughout, the medical device delivers the therapeutic agent to a depth below the surface of the skin of from about 50 μm to about 4000 μm, from about 250 μm to about 2000 μm, or from about 350 μm to about 1000 μm. In certain embodiments, which may be combined with other embodiments disclosed throughout, each of the microneedles in the medical device has a length between about 200 to about 800 μm, between about 250 to about 750 μm, or between about 300 to about 600 μm.

In certain embodiments, which may be combined with other embodiments disclosed throughout, a therapeutic agent administered according to any of the disclosed methods comprises Enbrel.

In certain embodiments, which may be combined with other embodiments disclosed throughout, are provided a dose sparing amount or concentration of a therapeutic agent that is therapeutically effective for treating an arthritic disease or associated condition, or a symptom or associated with an arthritic disease or associated condition, upon administration via a medical device that administers the dose-sparing amount or concentration to the lymphatic system of a subject having, or suspected of having, the arthritic disease or associated condition.

### BRIEF DESCRIPTION OF THE DRAWINGS

FIG. 1 provides an illustration of human lymphatic architecture. The illustration shows initial lymphatic capillaries that are found below the stratum corneum and epidermis in the dermal papillae at the epidermal-dermal junction. These capillaries consist of highly fenestrated openings that allow for large molecules and modalities to be cleared from the extracellular space. Sofusa lymphatic delivery systems (SOFUSA) enable entry into the initial lymphatic capillaries, where lymph follows a unidirectional flow into collecting lymphatic vessels, then afferent lymphatic vessels that enter draining lymph nodes, followed by efferent lymphatic vessels that exit the lymph nodes.

FIG. 2 depicts an exemplary attachment and positioning scheme of an exemplary, wearable, Sofusa® Lymphatic Delivery System (SOFUSA) device strapped onto a subject's arm for lymphatic administration of therapeutic agents. In this exemplary depiction, the wearable device is activated with an applicator to facilitate needle penetration into the subject's skin, whereby the therapeutic agent is slowly infused from a syringe pump into the subjects's arm over a period of time based on the desired dose.

FIG. 3 illustrates an exemplary microneedle array with nanotopographical film. FIG. 3A provides a schematic representation of the transdermal microneedle device used in the Examples. An impermeable backing (tan), drug reservoir (green), rate-controlling membrane (yellow), and silicon microneedle array (MNA) (gray) are shown. Microneedles are designed with precise dimensions. Drug flow from the reservoir down the grooves of the microneedles is indicated by a green dashed arrow. Perforations are denoted with a white arrowhead. FIG. 3B provides a scanning electron microscopy (SEM) image of the microneedle array. FIG. 3C provides an SEM image of a single microneedle. FIG. 3D provides an SEM image of a single microneedle depicting nanostructure coated onto each microneedle.

FIG. 4 provides a graphic depiction of DAS28 Disease Activity Scores determined every two weeks throughout the studies provided in the Examples. FIG. 4A and FIG. 4B provide DAS28 scores based on ESR and CRP, respectively, for Patient ID 01-002. FIG. 4C and FIG. 4D provide DAS28 scores based on ESR and CRP, respectively, for Patient ID 01-002 (as in FIG. 4A and FIG. 4B), Patient ID 01-004, and Patient ID 01-006.

FIG. 5 provides a graphic depiction of tender and swollen joint counts performed every two weeks throughout the studies provided in the Examples. FIG. 5A and FIG. 5B provide 28-count measurements and 68/66-count measurements, respectively, for Patient ID 01-002. FIG. 5C and FIG. 5B provide 68/66-count measurements and 28-count measurements, respectively, for Patient ID 01-002 (as in FIG. 5A and FIG. 5B), Patient ID 01-004, and Patient ID 01-006.

FIG. 6 provides a comparison of lymphatic function before and six weeks after SOFUSA-mediated Enbrel administration.

FIG. 7 provides a sectional view of an exemplary fluid delivery apparatus in a pre-use configuration.

FIG. 8 provides a sectional view of the fluid delivery apparatus in a pre-activated configuration.

FIG. 9 provides an exploded, sectional view of fluid delivery apparatus.

FIG. 10 provides a sectional view of a collet assembly of the fluid delivery apparatus.

FIG. 11 provides an exploded, perspective view of the collet assembly shown in FIG. 10.

FIG. 12 provides the average concentration of etanercept in lymph nodes when delivered via intravenous, subcutaneous, intradermal, or lymphatic delivery (e.g., via Sofusa delivery). Etanercept concentrations measured at 12 hours and 36 post administration, as indicated.

Unless otherwise indicated, the drawings provided herein are meant to illustrate features of embodiments of the disclosure or results of representative experiments illustrating some aspects of the subject matter disclosed herein. These features and/or results are believed to be applicable in a wide variety of systems comprising one or more embodiments of the disclosure. As such, the drawings are not meant to include all additional features known by those of ordinary skill in the art to be required for the practice of the embodiments, nor are they intended to be limiting as to possible uses of the methods disclosed herein.

### DETAILED DESCRIPTION

It has now been discovered, as described herein and throughout, that inter alia, lymphatic administration of a therapeutic agent to the lymphatic system of a subject having, or suspected of having, an arthritic disease or associated condition, or a symptom associated with an arthritic disease or associated condition, thereby treating the arthritic disease or associated condition. In certain embodiments, such benefits are achieved via SOFUSA-mediated administration or delivery of such therapeutic agents to such subjects. In certain embodiments, SOFUSA-mediated lymphatic administration of therapeutic agents, which may comprise anti-inflammatory agents, such as Enbrel, allow a substantially reduced amount or dose relative to a typical dose or amount of such a therapeutic agent when administered by a non-lymphatic route (such as a subcutaneous, intravenous, oral, buccal, rectal, or lingual route), to nonetheless constitute a therapeutically effective amount of the therapeutic agent, thereby treating the disease or associated condition and/or reducing one or more symptoms or clinical manifestations thereof using such substantially reduced amount or dose.

In certain embodiments, as described herein and throughout, the disclosed methods result in profound improvement in at least one symptom, clinical measure or index of efficacious therapy in subjects who are demonstrably poorly responsive, non-responsive or refractory to conventional therapy and administration routes (e.g., subcutaneous injection). Such SOFUSA-mediated lymphatic administration concomitantly resulted in improved lymphatic flow and pumping rate. In certain embodiments, such methods, and advantages that flow from their practice, are achieved via Sofusa Lymphatic Delivery System (SOFUSA)-mediated lymphatic delivery or administration of therapeutic agents, such as anti-inflammatory agents, including Enbrel. In certain embodiments such methods provide not only for enhanced therapeutic benefit in subjects having, or suspected of having an arthritic disease or associated condition, or one or more symptoms or clinical manifestations thereof, but provides for such benefit by administering a significantly lower dose relative to non-lymphatic administration or delivery routes. Accordingly, methods comprising SOFUSA-mediated administration of dose-sparing, therapeutically effective amounts or concentrations of therapeutic agents, such as inflammatory agents, including Enbrel, advantageously provides for treatment such arthritic diseases or associated conditions, and/or for reducing one or more symptoms or clinical manifestations thereof, while minimizing the propensity for undesired or untoward off-target or other side effects. Without wishing to be bound by any theory, such benefits are believed to result from an increased amount and/or concentration of the lymphatically administered therapeutic agents into and/or stimulated/increased in lymphatic flow pumping rate. This, in turn, is believed to facilitate flow of therapeutically effective amounts of the anti-inflammatory agent, which may be dose-sparing amounts or concentrations of such therapeutic agents to therapeutic target(s), tissues, immune cells, or regions of disease or injury, in order to provide therapeutic benefit.

Additional embodiments of the invention, examples of which are illustrated in the accompanying drawings, Examples, are provided herein and throughout. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims. The section headings used herein are for organizational purposes only and are not to be construed as limiting the desired subject matter in any way. In the event that any literature incorporated by reference contradicts any term defined in this specification, this specification controls.

In the following specification and the claims, reference will be made to a number of terms, which shall be defined to have the following meanings. The singular forms “a,” “an,” and “the” include plural references unless the context clearly dictates otherwise. The terms “comprising,” “including,” and “having” are intended to be inclusive and mean that there may be additional elements other than the listed elements. “Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise. Numeric ranges are inclusive of the numbers defining the range.

As used herein, positional terms such as upward, downward, upper, lower, top, bottom, and the like are used only for convenience to indicate relative positional relationships.

The terms “medicament”, “medication”, “medicine”, “therapeutic agent” and “drug” are used interchangeably herein and describe a pharmaceutical composition or product intended for the treatment of a disease or associated condition, and/or at least one symptom or clinical manifestation of such a disease or associated condition. The pharmaceutical composition or product will have a physiological effect on the patient when it is introduced into the body of a patient. The pharmaceutical composition can be in any suitable formulation unless a specific formulation type is required or disclosed. In some instances, the medicament will be approved by the US FDA while in other instances it may be experimental (e.g., clinical trials) or approved for use in a country other than the United States (e.g., approved for use in China or Europe). In instances where these terms are used, it is understood that they refer to both singular and plural instances. In some embodiments herein, two or more medicaments may be used in a form of combination therapy. In all cases, the selection of the proper medicament (singular or plural) will be based on the medical condition of the patient and the assessment of the medical professional administering, supervising and/or directing the treatment of the patient. Combination therapies are sometimes more effective than a single agent and used for many different medical conditions. It is understood that combination therapies are encompassed herein and envisioned with the subject matter disclosed.

An “effective amount” or a “therapeutically effective dose” in reference to a medicament is an amount sufficient to treat, ameliorate, or reduce the intensity of at least one symptom associated with the medical condition. In some aspects of this disclosure, an effective amount of a medicament is an amount sufficient to affect a beneficial or desired clinical result including alleviation or reduction in one or more symptoms of a medical condition. In some embodiments, an effective amount of the medicament is an amount sufficient to alleviate all symptoms of a medical condition. In some aspects, a dose of the therapeutic agent will be administered that is not therapeutically effective by itself. In these aspects, multiple doses may be administered to the patient either sequentially (using the same device or different devices) or simultaneously such that the combination of the individual doses is therapeutically effective. For simultaneous administration, additional medical devices comprising a plurality of microneedles or an entirely different route of administration may be used.

The term “dose-sparing amount” or “dose-sparing concentration” means an amount or concentration of an agent that is therapeutically effective when provided to a subject via lymphatic delivery or administration in accordance with the methods disclosed herein and throughout, but is not therapeutically effective (or is less therapeutically effective) when administered via non-lymphatic administration routes (such as subcutaneous, intravenous, intramuscular, oral, lingual, sublingual, buccal, etc.)

The term “subject” or “patient”, used interchangeably herein, means a warm-blooded animal such as a mammal which is the subject of treatment for a disease or condition that causes at least one symptom. It is understood that at least humans, dogs, cats, and horses are within the scope of the meaning of the term. In certain embodiments, the subject is human.

As used herein, the terms “distal” and “proximal” are used in their anatomical sense. Distal means a given position or structure is situated farther from the center of the body or point of attachment of the limb when compared to another position or structure. Proximal is the opposite of distal. Proximal means a given position or structure is situated closer to the center of the body or point of attachment of the limb when compared to another position or structure. For example, the wrist is distal to the elbow and the shoulder is proximal to the elbow.

As used herein, the term “treat” or “treatment”, or a derivative thereof, contemplates partial or complete reduction or amelioration, or prevention, of at least one symptom associated with the medical condition of the patient. “Prevention” or “preventing” means a medical condition from occurring (e.g., an inflammatory or autoimmune condition) is considered a form of treatment. “Reducing” the incidence of a medical condition (e.g., an inflammatory or autoimmune condition) is considered a form of treatment.

Etanercept is a fusion protein produced by recombinant DNA and is sold under the trade name of Enbrel®. Etanercept fuses the TNF receptor to the constant region of an IgGI antibody, and, when administered to a patient, reduces the biological effect of TNF present in the patient. As such, it is considered a TNF inhibitor. In the United States, Etanercept has been approved for clinical use by the FDA for the treatment of moderate to severe rheumatoid arthritis (RA), moderate to severe polyarticular juvenile rheumatoid arthritis (JRA), psoriatic arthritis, ankylosing spondylitis, and moderate to severe plaque psoriasis. Due to the serious number of secondary infections associated with Enbrel®, the FDA requires a black box warning—the most serious level of warning possible under current FDA guidelines. As used herein, the term etanercept and Enbrel® are used interchangeably and also encompass any biosimilars or bioequivalents thereof.

As used herein, “bioavailability”, means the total amount of a given dosage of the administered agent that reaches the blood compartment. This is generally measured as the area under the curve (AUC) in a plot of concentration vs. time.

As used herein, the phrase “side effects” encompasses unwanted, untoward, and/or adverse effects of a prophylactic or therapeutic agent. Adverse effects are always unwanted, but unwanted effects are not necessarily adverse. An adverse effect from a prophylactic or therapeutic agent might be harmful or uncomfortable or risky. Side effects from therapeutic agents include, for example, gastrointestinal toxicity such as, but not limited to, early and late-forming diarrhea and flatulence, nausea, vomiting, anorexia, leukopenia, anemia, neutropenia, asthenia, abdominal cramping, fever, pain, loss of body weight, dehydration, alopecia, dyspnea, insomnia, dizziness, mucositis, xerostomia, and kidney failure, as well as constipation, nerve and muscle effects, temporary or permanent damage to kidneys and bladder, flu-like symptoms, unwanted or excessive immunosuppression, unwanted or excessive immune activation, cytokine release syndrome or cytokine storm, fluid retention, temporary or permanent infertility, fatigue, dry mouth, loss of appetite, rashes or swellings at the site of administration, flu-like symptoms such as fever, chills and fatigue, digestive tract problems, allergic reactions, depression, eye problems, weight fluctuation. Additional undesired effects typically experienced by patients are numerous and known in the art, see, e.g., the Physicians' Desk Reference (69th ed., 2015), which is incorporated herein by reference in its entirety.

Cₘₐₓ refers to the maximum concentration that a medicament achieves in the plasma or tissue of a patient after the medicament has been administered while Ct refers to the concentration that a medicament achieves at a specific time (t) following administration. Unless otherwise stated, all discussion herein is in regard to pharmacokinetic parameters in plasma.

The AUCₜ refers to the area under the plasma concentration time curve from time zero to time t following administration of the medicament.

The AUC_{∞} refers to the area under the plasma concentration time curve from time zero to infinity (infinity meaning that the plasma concentration of the medicament is below detectable levels).

Tₘₐₓ is the time required for the concentration of a medicament to reach its maximum blood plasma concentration in a patient following administration. Some forms of administration of a medicament will reach their Tₘₐₓ slowly (e.g., tablets and capsules taken orally) while other forms of administration will reach their Tₘₐₓ almost immediately (e.g., subcutaneous and intravenous administration).

“Steady state” refers to the situation where the overall intake of a drug is approximately in dynamic equilibrium with its elimination.

A discussion of various pharmacokinetic parameters and the methods of measuring and calculating them can be found in Clinical Pharmacokinetics and Pharmacodynamics: Concepts and Applications, M. Rowland and T. N. Tozer, (Lippincott, Williams & Wilkins, 2010) which is incorporated by reference for its teachings thereof.

“Or” is used in the inclusive sense, i.e., equivalent to “and/or,” unless the context requires otherwise.

In certain embodiments, provided are exemplary devices and methods for delivering a therapeutic effective amount of a therapeutic agent to a subject having, or suspected of having, an arthritic disease or associated condition, or a symptom associated with an arthritic disease or associated condition, to the lymphatic system of the subject. In certain embodiments, a Sofusa Lymphatic Delivery System (SOFUSA) device is employed in accordance with the methods disclosed herein and throughout. Exemplary such medical devices are disclosed, e.g., in WO 2011/135530, WO2011/135533, WO/2012/020332, WO 2014/132239, WO 2014/188343, WO 2015/168210, WO 2015/168215, WO 2015/168217, WO 2015/168219, WO2017/0189258, WO 2017/0189259, WO 2017/019535, WO 2018/111611, WO 2018/111616, and/or WO 2018/111621.

In certain embodiments, the therapeutic target for the therapeutic agent is identified, and a medical device, such as a SOFUSA device is placed such that the therapeutic agent is administered to the lymphatic system of the subject such that a therapeutically effective amount of the therapeutic agent is carried by the lymph vessels to that target. In other embodiments, the therapeutic target or exact location of the therapeutic target may be unknown or less clearly defined, and the therapeutic agent is administered, via a medical device, such as SOFUSA device as described above and throughout, into the lymphatic system of such a subject, and the therapeutic agent is intended to traverse the lymphatic system to either the right lymphatic duct or the thoracic duct. The therapeutic agent then enters the circulatory system of the patient leading to systemic exposure to the agent. For certain arthritic diseases or associated conditions for which a specific therapeutic target for delivery of the therapeutic agent is unknown, a therapeutic agent administered via a medical device, such as a SOFUSA device as described above and throughout, may traverse certain lymph nodes before reaching either of the draining ducts; such administration is considered to result in systemic exposure. As such, the skilled artisan, in view of the present disclosure can apply methods and devices disclosed herein to provide targeted, regional administration of a therapeutic agent or more widespread systemic administration. A skilled artisan may also determine which mode of administration is appropriate for an individual subject and place the medical device or devices accordingly.

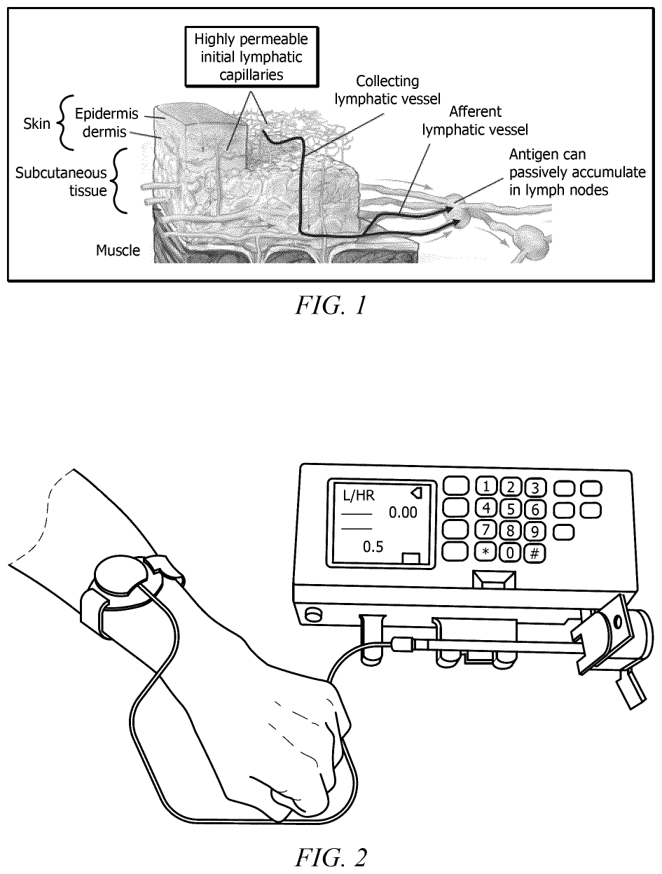

In certain embodiments, SOFUSA is employed to access the lymphatic system directly through the skin at the epidermal/dermal boundary (See FIG. 1) thereby providing assess of a therapeutic agent to afferent lymphatic capillaries. In certain embodiments, microneedle design of SOFUSA enables large molecules and biologics to be delivered through the skin. In certain embodiments, the hollow microneedles of SOFUSA penetrate the stratum corneum and epidermis skin layers so that a therapeutically effective amount of a therapeutic agent, such as an anti-inflammatory agent, such as Enbrel, can be infused at the epidermal-dermal interface. In certain embodiments, at this interface, the microneedles deliver a greater amount or concentration of such a therapeutic agent into the initial lymphatic vessels than that achieved with non-lymphatic administration routes, such as subcutaneous, intravenous administration, oral, buccal, lingual, or other non-lymphatic routes, thereby providing substantial and advantageous improvements in the response rate, magnitude, and/or duration. Without wishing to be bound by any theory, it is believed that the methods of lymphatic administration of therapeutic agents disclosed herein and throughout facilitates delivery of sufficient amounts of the therapeutic agents by lymph vessels to the therapeutic target, tissue, or cells, such as immune cells.

In certain embodiments, such therapeutic target(s) may comprise, e.g., one or more inflamed joints other source or clinical manifestation of an arthritic disease or associated condition in a subject having, or suspected of having, such an arthritic disease or associated condition. In certain embodiments, while some systemic exposure will occur, the administration is much more regionalized.

In some aspects, the therapeutic target(s) is or comprises a lymph node, a lymph vessel, an organ that is part of the lymphatic system, or a combination thereof. In some aspects, the therapeutic target is a lymph node. In some aspects, the therapeutic target is a specific lymph node as described herein and throughout.

In some embodiments, delivery of the therapeutic agent to the lymphatic system is delivery into the vessels of the lymphatic vasculature, the lymph nodes as described herein and throughout. In some aspects, delivery is to the superficial lymph vessels. In yet another aspect, delivery is to one or more lymph nodes. The specific target for delivery will be based on the medical needs of the patient.

In patients where more than one medical device is used to deliver the therapeutic agent to a plurality of locations on the body of a patient, the overall dose of the therapeutic agent at each location must be carefully adjusted such that the patient does not receive an overall unsafe combined dose of the agent. Being able to more selectively target specific locations in or on the body of a patient more precisely often means a lower dose is required at each specific location. In some embodiments, the dose administered to target one or more locations on the body of a patient is lower than a dose administered by other routes, including intravenous and subcutaneous administration.

Because the lymph fluid circulates throughout the body of a patient in a similar manner to blood in the circulatory system, any single position in the lymphatic vasculature can be upstream or downstream relative to another position. As used herein in reference to the lymphatic vasculature, the term “downstream” refers to a position in the lymphatic system closer (as the fluid travels through the vessels in a healthy patient) to either the right lymphatic duct or the thoracic duct relative to the reference position (e.g., a tumor or internal organ or a joint). As used herein, the term “upstream” refers to a position in the lymphatic system that is farther from the right lymphatic duct or the thoracic duct relative to the reference position. Because the direction of fluid flow in the lymphatic system can be impaired or reversed due to the medical condition of the patient, the terms “upstream” and “downstream” do not specifically refer to the direction of fluid flow in the patient undergoing medical treatment. They are positional terms based on their physical position relative to the draining ducts as described.

Because lymph nodes often occur in a group as opposed to being present as a single isolated node, the term “lymph node” as used herein can be singular or plural and refer to either a single isolated lymph node or a group of lymph nodes in a small physical location. For example, a reference to the inguinal lymph node or inguinal lymph nodes refers to the group of lymph nodes that are recognized by a person skilled in the art (i.e., a medical professional such as a doctor or a nurse) as a group of lymph nodes located in the hip/groin area or femoral triangle in a patient. It also refers to both the superficial and deep lymph nodes unless specifically stated otherwise.

In some embodiments, the lymph node is selected from the group consisting of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, and splenic lymph nodes, and combinations thereof.

In some embodiments, two or more different lymph nodes are selected. In some embodiments, three or more different lymph nodes are selected. The lymph nodes may be on either side of the body of the patient. In yet another embodiment, the lymph node is the inguinal lymph node. The inguinal lymph node may be the right inguinal lymph node, the left inguinal lymph node or both. In yet another embodiment, the lymph node is the axillary lymph node. The axillary lymph node may be the right axillary lymph node, the left axillary lymph node or both.

In some embodiments, two or more different lymph nodes are selected. In some embodiments, three or more different lymph nodes are selected. The lymph nodes may be on either side of the body of the patient. In yet another embodiment, the lymph node is the inguinal lymph node. The inguinal lymph node may be the right inguinal lymph node, the left inguinal lymph node or both. In yet another embodiment, the lymph node is the axillary lymph node. The axillary lymph node may be the right axillary lymph node, the left axillary lymph node or both.

In some embodiments, the medicament is delivered to the interstitium of the patient, e.g., to a space between the skin and one or more internal structures, such as an organ, muscle, or vessel (artery, vein, or lymph vessel), or any other spaces within or between tissues or parts of an organ. In still yet another embodiment, the medicament is delivered to both the interstitium and the lymphatic system. In embodiments where the therapeutic agent is delivered to the interstitium of the patient, it may not be necessary to locate the lymph nodes or lymphatic vasculature of the patient before administering the therapeutic agent.

One embodiment disclosed herein is a method for administering a therapeutic agent to the lymphatic system of a patient. The method generally comprises placing a first medical device comprising a plurality of microneedles on the skin of the patient at a first location proximate to a first position under the skin of the patient, wherein the first position is proximate to lymph vessels and/or lymph capillaries that drain into the right lymphatic duct, and wherein the microneedles of the first medical device have a surface comprising nanotopography; placing a second medical device comprising a plurality of microneedles on the skin of the patient at a second location proximate to a second position under the skin of the patient, wherein the second position is proximate to lymph vessels and/or lymph capillaries that drain into the thoracic duct, and wherein the microneedles of the second medical device have a surface comprising nanotopography; inserting the plurality of microneedles of the first medical device into the patient to a depth whereby at least the epidermis is penetrated and an end of at least one of the microneedles is proximate to the first position; inserting the plurality of microneedles of the second medical device into the patient to a depth whereby at least the epidermis is penetrated and an end of at least one of the microneedles is proximate to the second position; and administering via the microneedles of the first medical device a first dose of the therapeutic agent into the first position; administering via the microneedles of the second medical device a second dose of the therapeutic agent into a second position; wherein administering the doses cumulatively provides a therapeutically effective amount of the therapeutic agent.

In another aspect, disclosed herein is a method for administering a therapeutic agent to the lymphatic system of a patient. The method generally comprises placing a first medical device comprising a plurality of microneedles on the skin of the patient at a first location proximate to a first position under the skin of the patient, wherein the first position is proximate to lymph vessels and/or lymph capillaries that drain into the right lymphatic duct, and wherein the microneedles of the first medical device have a surface comprising nanotopography; placing a second medical device comprising a plurality of microneedles on the skin of the patient at a second location proximate to a second position under the skin of the patient, wherein the second position is proximate to lymph vessels and/or lymph capillaries that drain into the thoracic duct, and wherein the microneedles of the second medical device have a surface comprising nanotopography; inserting the plurality of microneedles of the first medical device into the patient to a depth whereby at least the epidermis is penetrated and an end of at least one of the microneedles is proximate to the first position; inserting the plurality of microneedles of the second medical device into the patient to a depth whereby at least the epidermis is penetrated and an end of at least one of the microneedles is proximate to the second position; administering via the microneedles of the first medical device a first therapeutically effective dose of the therapeutic agent into the first position; and administering via the microneedles of the second medical device a second therapeutically effective dose of the therapeutic agent into the second position; wherein a beginning time for administering the first dose and the second dose are different and separated by a period of time.

In some aspects disclosed herein, the first position and second position are reversed and the first position is proximate to lymph vessels and/or lymph capillaries that drain into the thoracic duct and the second position is proximate to lymph vessels and/or lymph capillaries that drain into the right lymphatic duct. As noted, one medical device drains into one of the two draining ducts in the lymphatic system while the other medical device drains into the other draining duct. This method is envisioned to administer at least a therapeutic agent to the lymphatic system of the patient such that different parts of the lymphatic system are exposed to the therapeutic agent. In some aspects, two or more medical devices are placed such that they drain into the same draining duct but they target different regions of the lymphatic system of the patient. For example, one device may be placed on the left arm of the patient and one device may be placed on the left leg of the patient. Although the therapeutic agent would ultimately drain through the same duct for site of administration, the therapeutic agent would traverse significantly different regions of the lymphatic system of the patient.

In some aspects, the first dose of the therapeutic agent and the second dose of the therapeutic agent are not therapeutically effective individually, but the combined amount of the doses is therapeutically effective. The first dose and the second dose can be administered sequentially or simultaneously. In some aspects, the first dose and the second dose are administered sequentially. In some aspects, the first dose and the second dose are administered simultaneously. In some aspects, administration of the two doses at least partially overlaps in time. This means that the administration of the two doses commences at different times, but the administration of the second dose begins before the administration of the first dose ends.

The location on the body of the patient is selected based on the medical condition of the patient and the knowledge of the medical professional supervising, directing and/or administering the treatment. For each medical device used with the methods disclosed herein, the location of the medical device on the body of the patient is selected independently of the other medical devices with the caveat that the objective of this method is to expose different parts of the lymphatic system to the therapeutic agent. In some aspects, each medical device is placed on a limb (i.e., arm or leg) of the patient. In order to achieve maximum exposure of the lymphatic system to the therapeutic agent, one device is placed on the right arm of the patient while the other device is place on the left leg of the patient. Alternatively, one device could be placed on the left arm of the patient while the other device is placed on the right leg of the patient. In yet another aspect, one medical device is placed on the right arm of the patient while the other medical device is placed on either the left arm or left leg of the patient. In yet another aspect, one medical device is placed on the left arm of the patient and the other medical device is placed on the right arm or right leg of the patient. A device on the arm of the patient may be located proximate to the wrist or hand of the patient while a device on the patient may be located proximate to the ankle or foot of the patient.

In still yet another aspect, the methods disclosed herein further comprise placing a third medical device comprising a plurality of microneedles on the skin of the patient at a third location proximate to a third position under the skin of the patient, wherein the third position is proximate to lymph vessels and/or lymph capillaries; inserting the plurality of microneedles of the third medical device into the patient to a depth whereby at least the epidermis is penetrated and an end of at least one of the microneedles is proximate to the third position; and administering via the third medical device a third dose of said therapeutic agent; and wherein the third location is different than the first location and the second location, and the third position is different that the first position and the second position.

In still yet another aspect, the methods disclosed herein further comprise placing a fourth medical device comprising a plurality of microneedles on the skin of the patient at a fourth location proximate to a fourth position under the skin of the patient, wherein the fourth position is proximate to lymph vessels and/or lymph capillaries; inserting the plurality of microneedles of the fourth medical device into the patient to a depth whereby at least the epidermis is penetrated and an end of at least one of the microneedles is proximate to the fourth position; and administering via the fourth medical device a fourth dose of said therapeutic agent; and wherein the first location, the second location, the third location, and the fourth location are on different limbs of the patient.

For any of the methods disclosed in, including those that use two medical devices, three medical devices, or four medical devices, in some aspects, each medical device is placed such that it initially drains into different lymph nodes, and wherein the draining lymph nodes are selected from the group of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, and splenic lymph nodes.

In one non-limiting example where three medical devices are used on a patient, the first device is placed on the right forearm of the patient which would then drain into the right axillary lymph nodes; the second device is placed on the left forearm of the patient which would then drain into the left axillary lymph nodes; and the third device is placed on the left thigh of the patient which would then drain into the left inguinal lymph nodes. In this instance the second and third devices would both drain into the thoracic duct but the initial draining lymph nodes are different.

In some aspects, the first dose of the therapeutic agent, the second dose of the therapeutic agent, and if present, the third dose of the therapeutic agent and the fourth dose of the therapeutic agent may each be administered to the patient sequentially or simultaneously. Doses may be combined such that the first and second dose are administered simultaneously while the third and fourth dose are administered together but sequentially relative to the first and second doses. In another aspect, the first and third dose and simultaneously administered while the second and fourth dose are administered simultaneous with each other and sequentially with the first and third dose. In yet another aspect, each dose is administered sequentially.

For any individual dose or combination of doses that are administered sequentially, there is a predetermined period of time between the beginning of each administration. That predetermined period of time may be 15 minutes, 30 minutes, 45 minutes, 1 hour, 2 hours, 3 hours, 4 hours, 5 hours, 6 hours, 7 hours, 8 hours, 12 hours, 16 hours, 20 hours, 24 hours, 36 hours, 48 hours, 60 hours, or 72 hours. The predetermined period may be from about 15 minutes to about 72 hours or a time increment therebetween. Each period of time is selected independently of any other period of time and is based on the medical needs of the patient and the assessment of the medical professional administering, supervising or directing the treatment of the patient. Because the time that it takes to administer a dose of the therapeutic agent with the medical device is not zero, it is possible that the initiation of administering a subsequent dose of the therapeutic agent will be before the completion of the administration of the prior dose. For example, the administration of the second dose of the therapeutic agent may begin before the administration of the first dose of the therapeutic agent is complete. In yet another aspect, the predetermined period of time is based on the ending of one dose and the initiation of the next dose.

In some aspects, the therapeutic agent is effective in treating or relieving one or more symptoms or clinical manifestations of an arthritic disease or associated condition in a subject having, or suspected of having, such an arthritic disease or associated condition. In some aspects, the therapeutic agent is an antibody that inhibits TNF-α. In some embodiments, the therapeutic agent is adalimumab, adalimumab-atto, certolizumab pegol, etanercept, etanercept-szzs, golimumab, infliximab, infliximab-dyyb, or a variant, analog, biosimilar or bioequivalent of any one of the foregoing agents. In some embodiments, the therapeutic agent is etanercept or a variant, analog, biosimilar thereof, or bioequivalent thereof. In some embodiments, the therapeutic agent is adalimumab or a variant, analog, biosimilar thereof, or bioequivalent thereof. In some embodiments, the therapeutic agent is an immune-suppressing agent. In certain embodiments, the immune-suppressing agent is adalimumab (Humira®), etanercept (Enbrel®), infliximab (Remicade®), ustekinumab (Stelara®), rituximab (Rituxan®), secukinumab (Cosentyx®), omalizumab (Xolair®), natalizumab (Tysabri®), ixekizumab (Taltz®), obinutuzumab (Gazyva®), or rituximab/hyaluronidase human (Rituxan Hycela™) or a variant, analog, biosimilar, or bioequivalent of any of the foregoing.

In still yet another embodiment, disclosed herein is a method for increasing the bioavailability of a therapeutic agent in a patient, the method comprising placing at least one medical device that comprises a plurality of microneedles on the skin surface of the patient; and administering a therapeutic agent with the at least one medical device to the patient.

In some embodiments, the methods for delivering a therapeutic agent to a patient as described herein result in an equivalent blood serum absorption rate of one or more therapeutic agents described herein as compared to intravenous, subcutaneous, intramuscular, intradermal or parenteral delivery routes while retaining relatively higher rates of lymphatic delivery as described herein. Without being bound by any theory, the rate of delivery and increased bioavailability may be due to the lymphatic circulation of one or more agents through the thoracic duct or the right lymphatic duct and into the blood circulation. Standard highly accurate and precise methodologies for measuring blood serum concentration and therapeutic monitoring at desired time points may be used that are well known in the art, such as radioimmunoassays, high-performance liquid chromatography (HPLC), fluorescence polarization immunoassay (FPIA), enzyme immunoassay (EMIT) or enzyme-linked immunosorbant assays (ELISA). For calculating the absorption rate using the methods described above, the drug concentration at several time points should be measured starting immediately following administration and incrementally thereafter until a Cₘₐₓ value is established and the associated absorption rate calculated.

One embodiment disclosed herein is a method for treating an inflammatory medical condition in a patient. The method generally comprises locating at least one inflammatory locus in the patient, wherein the at least one inflammatory locus comprises lymph vessels, lymph capillaries, lymph nodes, lymph organs or any combination thereof; locating a first position in the lymphatic system of the patient that is upstream of the inflammatory locus; placing a medical device comprising a plurality of microneedles on the skin of the patient proximate to the first position, and wherein the microneedles have a surface comprising nanotopography; inserting the plurality of microneedles into the patient to a depth whereby at least the epidermis is penetrated; and administering via the plurality of microneedles to the first position a therapeutically effective amount of an antibody that inhibits TNF-α or etanercept or a biosimilar or bioequivalent thereof.

In another aspect, disclosed herein is a method for lowering the TNF-α level in a patient. The method generally comprises locating a first position in the lymphatic system of the patient; placing a medical device comprising a plurality of microneedles on the skin of the patient proximate to the first position, and wherein the microneedles have a surface comprising nanotopography; inserting the plurality of microneedles into the patient to a depth whereby at least the epidermis is penetrated; and administering via the plurality of microneedles to the first position a therapeutically effective amount of an antibody that inhibits TNF-α or etanercept or a biosimilar or bioequivalent thereof.

In another aspect, disclosed herein is a method for treating an inflammatory medical condition in a patient. The method generally comprises locating at least one inflammatory locus in the patient comprising lymph nodes, lymph capillaries, lymph vessel, lymph organs or any combination thereof; placing a medical device comprising a plurality of microneedles on the skin of the patient proximate to a first position under the skin of the patient, wherein the first position is situated such that it comprises selected lymph capillaries and/or lymph vessels that deliver lymph directly into the lymphatic system in the inflammatory locus, and wherein the microneedles have a surface comprising nanotopography; inserting the plurality of microneedles into the patient to a depth whereby at least the epidermis is penetrated; and administering via the plurality of microneedles to the selected lymph capillaries and/or lymph vessels of the patient a therapeutically effective amount of an antibody that inhibits TNF-α or etanercept or a biosimilar or bioequivalent thereof.

In some embodiments, a method for treating an inflammatory medical condition in a patient is provided. The method comprises placing a medical device comprising a plurality of microneedles on the skin of the patient proximate to a first position under the skin of the patient, wherein the first position is situated such that it comprises lymph capillaries and/or lymph vessels that deliver lymph directly into the lymphatic system, and wherein the microneedles have a surface comprising nanotopography; inserting the plurality of microneedles into the patient to a depth whereby at least the epidermis is penetrated; and administering via the plurality of microneedles to the lymph capillaries and/or lymph vessels of the patient an antibody that inhibits TNF-α or etanercept or a biosimilar or bioequivalent thereof, thereby treating the inflammatory medical condition.

In some embodiments, a method disclosed herein comprising administering an antibody that inhibits TNF-α or etanercept or a biosimilar or bioequivalent thereof has any of the features set forth above with respect to methods of administering a therapeutic agent to the lymphatic system of a patient, e.g., including administration into at least a first position that is proximate to lymph vessels and/or lymph capillaries that drain into the right lymphatic duct and the thoracic duct, respectively.

In some embodiments, the associated condition is an autoimmune condition. In certain embodiments, the associated condition is an autoimmune condition is selected from the group consisting of Behçet's disease, sarcoidosis, rheumatoid arthritis (RA), juvenile arthritis, psoriatic arthritis, plaque psoriasis, hidradenitis suppurativa, autoimmune uveitis, ankylosing spondylitis, ulcerative colitis (UC), Crohn's disease, and combinations thereof. In some embodiments, the inflammatory medical condition is rheumatoid arthritis. In some embodiments, the inflammatory medical condition is psoriatic arthritis. In some embodiments, the inflammatory medical condition is plaque psoriasis. In some embodiments, the inflammatory medical condition is ulcerative colitis. In some embodiments, the inflammatory medical condition is Crohn's disease. The inflammatory medical condition may be acute or chronic.

The inflammatory locus in the patient can be any location in the patient that exhibits signs of inflammation; such signs include, but are not limited to, redness, swelling, fluid retention, joint pain, joint stiffness, unusual warmth at the location, and loss of joint function.

In some embodiments, administering is done to the lymph vessels upstream to inflammatory locus. In other embodiments, administering is done to both the lymph nodes and lymph vessels upstream of the inflammatory locus. In some aspects, it may not be necessary to locate a lymph node upstream of the inflammatory locus before administering the antibody that inhibits TNF-α or etanercept or a biosimilar or bioequivalent thereof to the patient.

Because some medical conditions can damage the lymphatic system of a patient, the flow of fluid in the lymphatic system can be impaired or even reversed (called backflow). This can lead to swelling in the surrounding tissues and organs of the patient. In some aspects, the medical device is placed such that backflow in the lymphatic system transports the antibody that inhibits TNF-α or etanercept or a biosimilar or bioequivalent thereof to the targeted location. For example, in a properly functioning lymphatic system, the downstream position relative to an inflammatory locus would not transport the antibody that inhibits TNF-α or etanercept or a biosimilar or bioequivalent thereof directly into the inflammatory locus. However, in an impaired lymphatic system, backflow from a downstream position relative to the inflammatory locus would transport the antibody that inhibits TNF-α or etanercept or a biosimilar or bioequivalent thereof directly to the inflammatory locus. A medical professional skilled in the art understands the manner by which the lymphatic system functions and will make treatment decisions for the patient based on that knowledge.

In some aspects, the inflammatory locus is a joint, a lymph node, a lymph vessel, an organ that is part of the lymphatic system or a combination thereof. In some aspects the therapeutic target is a joint. In some aspects, the therapeutic target is a lymph node. In some aspects, the therapeutic target is a specific lymph node as described elsewhere herein.

In some aspects the inflammatory locus is a joint selected from the group consisting of an ankle joint, a knee joint, a hip joint, a shoulder joint, an elbow joint, a metacarpophalangeal joint of the hands, a metatarsophalangeal joint in a foot, a wrist joint, a joint in the neck, and combinations thereof. In some aspects, the inflammatory locus is a psoriatic lesion.

In some aspects, the inflammatory locus is a knee, and the selected lymph capillaries and/or vessels flow into the popliteal lymph nodes. In some aspects, the inflammatory locus is a knee, and relative to the knee, the selected lymph capillaries and/or vessels are located distal to the heart.

In some aspects, the inflammatory locus is the neck, and the selected lymph capillaries and/or vessels flow into the cervical lymph nodes. In some aspects, the inflammatory locus is the neck, and, relative to the neck, the selected lymph capillaries and/or vessels are located distal to the heart.

In some aspects, the inflammatory locus is a shoulder, and the selected lymph capillaries and/or vessels flow into the pectoral lymph nodes, the superclavical lymph nodes, the axillary lymph nodes or any combination thereof. In some aspects, the inflammatory locus is a shoulder, and, relative to the shoulder, the selected lymph capillaries and/or vessels are located distal to the heart.

In some aspects, the inflammatory locus is an elbow, and the selected lymph capillaries and/or vessels flow into the epitrochlear lymph nodes and/or brachial lymph nodes. In some aspects, the inflammatory locus is an elbow, and, relative to the elbow, the selected lymph capillaries and/or vessels are located distal to the heart.

In some aspects, the inflammatory locus is a hip, and the selected lymph capillaries and/or vessels flow into the inguinal lymph nodes and/or the pelvic lymph nodes. In some aspects, the inflammatory locus is a hip, and, relative to the hip, the selected lymph capillaries and/or vessels are located distal to the heart. In some aspects, the inflammatory locus is a hip, and, relative to the hip, the selected lymph capillaries and/or vessels are located proximate to the heart.

In some aspects, the inflammatory locus is a psoriatic lesion and the selected lymph capillaries share common lymph vessels and/or lymph capillaries immediately adjacent to and/or within the psoriatic lesion. In some aspects, the medical device is placed at a location on the skin of the patient having lymph capillaries and/or vessels that flow directly into the lymph nodes within and/or closest to the psoriatic lesion.

In some aspects, when two medical devices that comprise a plurality of microneedles are used, the first medical device administers a first antibody that inhibits TNF-α or etanercept or a biosimilar or bioequivalent thereof to selected lymph capillaries and/or vessels distal to the heart relative to the inflammatory locus, and the second medical device administers a second antibody that inhibits TNF-α or etanercept or a biosimilar or bioequivalent thereof to selected lymph capillaries and/or vessels proximal to the heart relative to the inflammatory locus. In some aspects, the first therapeutic agent and the second therapeutic agent are the same. In some aspects, the first therapeutic agent and the second therapeutic agent are different. In this case each individual dose administered by each medical device may be smaller than a therapeutically effective dose, but the combined dose administered by the two medical devices is therapeutically effective.

In some embodiments, delivery of the antibody that inhibits TNF-α or etanercept or a biosimilar or bioequivalent thereof to the lymphatic system is delivery into the vessels of the lymphatic vasculature, the lymph nodes as described elsewhere herein, or both. In some aspects, delivery is to the superficial lymph vessels. In yet another aspect, delivery is to one or more lymph nodes. The specific target for delivery will be based on the medical needs of the patient. In one nonlimiting example, if a joint in the patient shows signs of an acute arthritic flare associated with a chronic arthritic condition, then the medical device comprising a plurality of microneedles can be placed on the patient such that it delivers the antibody that inhibits TNF-α or etanercept or a biosimilar or bioequivalent thereof directly to that specific joint. Alternatively, the medical device can be placed upstream of the joint such that the antibody that inhibits TNF-α or etanercept or a biosimilar or bioequivalent thereof is delivered to the lymph vessels that feed into the targeted joint. In some embodiments, two or more medical devices are used to target two or more different locations in the lymphatic system of the patient.

The placement of the medical device is based on the medical condition of the patient and/or an assessment by a medical professional. In one nonlimiting example, in a patient suffering from an acute flare-up of rheumatoid arthritis in one specific joint (e.g., the knee or shoulder), the medical device is placed upstream to deliver the agent to the lymph vessels that flow into and/or toward the inflamed joint in order to more effectively target the specific location of the acute flare-up. Similarly, in another nonlimiting example, a patient with significant patches of psoriatic lesions could have two or more medical devices placed in different locations on their body that are upstream of the lesions thereby targeting the specific lesions more precisely.

In some aspects, the antibody that inhibits TNF-α or etanercept or a biosimilar or bioequivalent thereof is effective in treating or relieving the symptoms of an inflammatory medical condition. In some embodiments, the antibody that inhibits TNF-α or etanercept or a biosimilar or bioequivalent thereofs adalimumab, adalimumab-atto, certolizumab pegol, etanercept, etanercept-szzs, golimumab, infliximab, infliximab-dyyb, or a biosimilar or bioequivalent of any of the foregoing agents. In some embodiments, the therapeutic agent is etanercept, a biosimilar thereof, or a bioequivalent thereof. In some embodiments, the therapeutic agent is adalimumab, a biosimilars thereof, or a bioequivalent thereof.

It is understood that when multiple doses of a therapeutic agent are administered to a patient, each individual dose may not be therapeutically effective, but the combined doses are therapeutically effective. The combined doses that are therapeutically effective may be smaller than a therapeutically effective dose if the same therapeutic agent is administered by a different route (e.g., subcutaneous, intravenous, etc.).

Medical devices that comprise an array of microneedles suitable for use herein are known in the art. Particular exemplary structures and devices comprising a means for controllably delivering one or more agents to a patient are described in International Patent Application Publication Nos. WO 2014/188343, WO 2014/132239, WO 2014/132240, WO 2013/061208, WO 2012/046149, WO 2011/135531, WO 2011/135530, WO 2011/135533, WO 2014/132240, WO 2015/16821, and International Patent Applications PCT/US2015/028154 (published as WO 2015/168214 A1), PCT/US2015/028150 (published as WO 2015/168210 A1), PCT/US2015/028158 (published as WO 2015/168215 A1), PCT/US2015/028162 (published as WO 2015/168217 A1), PCT/US2015/028164 (published as WO 2015/168219 A1), PCT/US2015/038231 (published as WO 2016/003856 A1), PCT/US2015/038232 (published as WO 2016/003857 A1), PCT/US2016/043623 (published as WO 2017/019526 A1), PCT/US2016/043656 (published as WO 2017/019535 A1), PCT/US2017/027879 (published as WO 2017/189258 A1), PCT/US2017/027891 (published as WO 2017/189259 A1), PCT/US2017/064604 (published as WO 2018/111607 A1), PCT/US2017/064609 (published as WO 2018/111609 A1), PCT/US2017/064614 (published as WO 2018/111611 A1), PCT/US2017/064642 (published as WO 2018/111616 A1), PCT/US2017/064657 (published as WO 2018/111620 A1), and PCT/US2017/064668 (published as WO 2018/111621 A1), all of which are incorporated by reference herein in their entirety.

In some aspects of the embodiments described herein, the one or more therapeutic agents are administered by applying one or more medical devices to one or more sites of the skin of the patient. One nonlimiting example of a medical device comprising a plurality of microneedles that is suitable for use with all of the methods disclosed herein is the Sofusa™ drug delivery platform available from Sorrento Therapeutics, Inc.

In some embodiments, the medical device is placed in direct contact with the skin of the patient. In some embodiments, an intervening layer or structure will be between the skin of the patient and the medical device. For example, surgical tape or gauze may be used to reduce possible skin irritation between the medical device and the skin of the patient. When the microneedles extend from the apparatus, they will contact and, in some instances, penetrate the epidermis or dermis of the patient in order to deliver the medicament to the patient. The delivery of the medicament can be to the circulatory system, the lymphatic system, the interstitium, subcutaneous, intramuscular, intradermal or a combination thereof. In some embodiments, the medicament is delivered directly to the lymphatic system of the patient. In some aspects, the medicament is delivered to the superficial vessels of the lymphatic system.

The term “proximate” as used herein is intended to encompass placement on and/or near a desired therapeutic target. Placement of the medical device proximate to the therapeutic target results in the administered therapeutic agent entering the lymphatic system and traversing to the intended therapeutic target. Additionally, placement of the medical device may be such that the administered therapeutic agent is directly administered to the therapeutic target.

In some embodiments described herein, the methods comprising a medical device comprising a plurality of microneedles may comprise delivering one or more agents through a device comprising two or more delivery structures that are capable of penetrating the stratum corneum of the skin of a patient and obtaining a delivery depth and volume in the skin and controllably delivering one or more agents at the administration rates as described herein. The delivery structures may be attached to a backing substrate of the medical device and arranged at one or a plurality of different angles for penetrating the stratum corneum and delivering the one or more agents. In some aspects, described herein the backing substrate comprising the delivery structures may be in contact with the skin of a patient and may have a cylindrical, rectangular, or geometrically irregular shape. The backing substrate further comprises a two dimensional surface area that in some aspects may be from about 1 mm² to about 10,000 mm². In some aspects, the delivery structures may comprise any geometric shape (e.g., a cylindrical, rectangular or geometrically irregular shape). In addition, the delivery structures may comprise a length and cross sectional surface area. In some aspects, the delivery structures may have an overall length that is greater than a cross sectional diameter or width. In some other aspects, the delivery structures may have a cross sectional diameter or width greater than an overall length. In some aspects, the cross sectional width of each of the delivery structures may be from about 5 μm to about 140 μm and the cross sectional area may be from about 25 μm² to about 65,000 μm², including each integer within the specified range. In some embodiments, the length of each of the delivery structures may be from about 10 μm to about 5,000 μm, from about 50 to about 3,000 μm, from about 100 to about 1,500 μm, from about 150 to about 1,000 μm, from about 200 to about 800 μm, from about 250 to about 750 μm, or from about 300 to about 600 μm. In some aspects, the length of each of the delivery structures may be from about 10 μm to about 1,000 μm, including each integer within the specified range. The surface area and cross-sectional surface areas as described herein may be determined using standard geometric calculations known in the art.

The delivery structures described herein need not be identical to one another. A medical device having a plurality of delivery structures may each have various lengths, outer diameters, inner diameters, cross-sectional shapes, nanotopography surfaces, and/or spacing between each of the delivery structures. For example, the delivery structures may be spaced apart in a uniform manner, such as, for example, in a rectangular or square grid or in concentric circles. The spacing may depend on numerous factors, including height and width of the delivery structures, as well as the amount and type of an agent that is intended to be delivered through the delivery structures. In some aspects, the spacing between each delivery structure may be from about 1 μm to about 1500 μm, including each integer within the specified range. In some aspects, the spacing between each deliver structure may be about 200 μm, about 300 μm, about 400 μm, about 500 μm, about 600 μm, about 700 μm, about 800 μm, about 900 μm, about 1000 μm, about 1100 μm, about 1200 μm, about 1300 μm, about 1400 μm or about 1500 μm. About as used in this context, “about” means ±50 μm.

In some embodiments described herein, the medical device may comprise a needle array in the form of a patch. In some aspects, the array of needles are able to penetrate a most superficial layer of the stratum corneum and initially deliver one or more agents as described herein to at least a portion or all of the non-viable epidermis, at least a portion of or all of the viable epidermis, and/or at least a portion of the viable dermis of a subject and subsequently to the lymphatic system of the patient. These needles may further comprise nanotopography on the surface of the needle in a random or organized pattern. In some aspects, the nanotopography pattern may demonstrate fractal geometry.

In some embodiments, the delivery structures may comprise an array of needles in fluid connection with a liquid carrier vehicle comprising one or more agents. In some aspects, the needles are microneedles. In some aspects, the array of needles may comprise between 2 and 50,000 needles with structural means for controlling skin penetration and fluid delivery to the skin (e.g., penetrating and delivering to the skin), see e.g., International Patent Application PCT/US2017/064668 (published as WO 2018/111621 A1), which is incorporated by reference herein in its entirety. In some other aspects, the array of needles may further comprise a manufactured random or structured nanotopography on each needle. The needle or needle array may be attached to a larger drug delivery apparatus comprising fluidic delivery rate controls, adhesives for attaching to the skin, fluidic pumps, and the like. If desired, the rate of delivery of the agent may be variably controlled by the pressure-generating means. Desired delivery rates as described herein to the epidermis may be initiated by driving the one or more agents described herein with the application of pressure or other driving means, including pumps, syringes, pens, elastomer membranes, gas pressure, piezoelectric, electromotive, electromagnetic or osmotic pumping, or use of rate control membranes or combinations thereof.

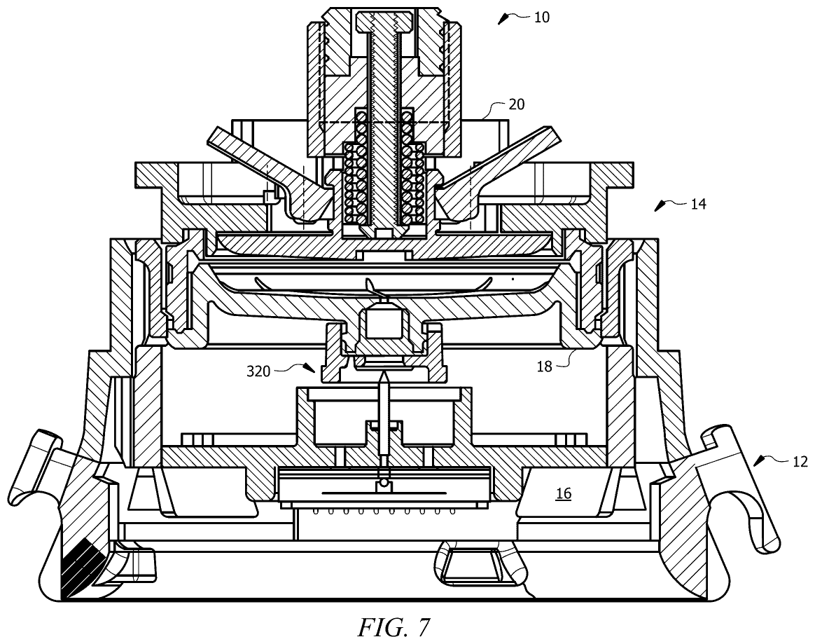

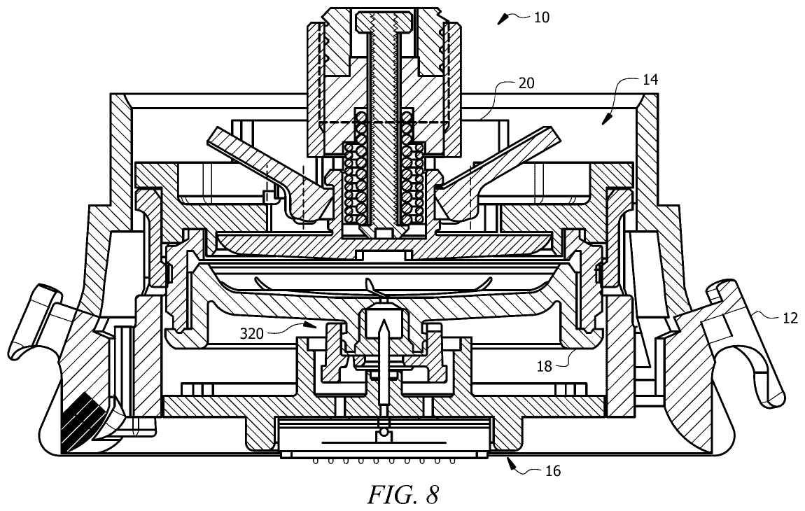

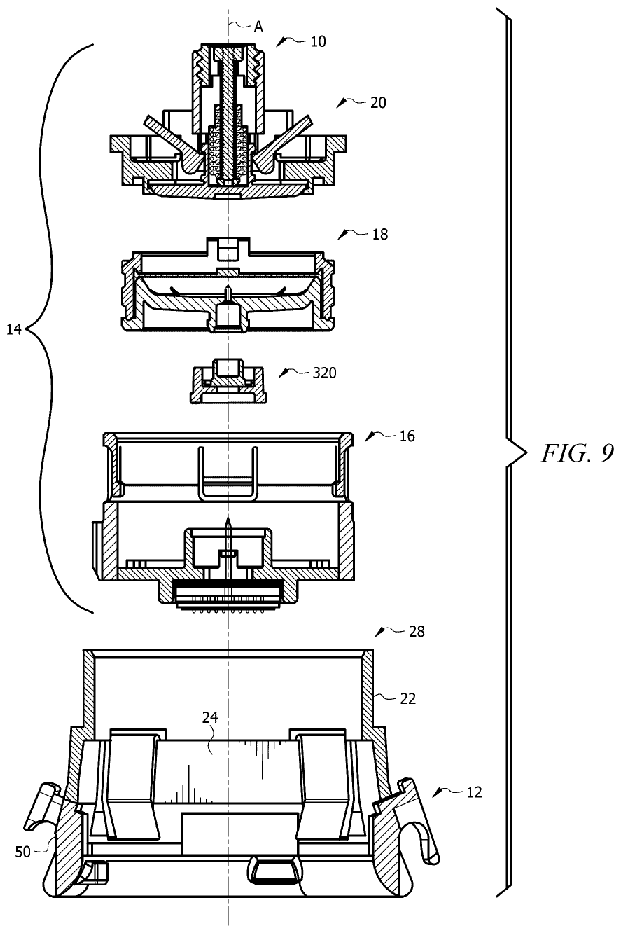

FIG. 7 is a sectional view of one exemplary example of a medical device comprising a plurality of microneedles (e.g., a medicament delivery apparatus), indicated generally by 10, in a pre-use configuration. It is understood that this example is suitable for use with all embodiments and aspects of the subject matter disclosed herein. Other devices as are known in the art are also suitable for use herewith. FIG. 8 is a sectional view of the fluid delivery apparatus 10 in a use configuration. FIG. 9 is an exploded, sectional view of fluid delivery apparatus 10. In the exemplary embodiment, the fluid delivery apparatus 10 includes a plurality of subassembly components coupled together to form the fluid delivery apparatus 10, including a collet assembly 12 and a fluid distribution assembly 14. The collet assembly 12 and the fluid distribution assembly 14 are indicated generally by their respective reference numbers. As shown in FIG. 9, the fluid distribution assembly 14 includes a plurality of additional subassembly components, including a plenum assembly 16, a cartridge assembly 18, a cap assembly 320, and a mechanical controller assembly 20. Each of the collet assembly 12, the fluid distribution assembly 14, the plenum assembly 16, the cartridge assembly 18, the cap assembly 320, and the mechanical controller assembly 20 is indicated generally in the accompanying drawings by their reference numbers. The collet assembly 12 forms the body or housing of the fluid delivery apparatus 10 and is slidably coupled to the fluid distribution assembly 14. To form the fluid distribution assembly 14, the cap assembly 320 is coupled to the cartridge assembly 18, and the cartridge assembly 18 is slidably coupled to the plenum assembly 16. In addition, the mechanical controller assembly 20, as explained in more detail below, is coupled to the cartridge assembly 18.

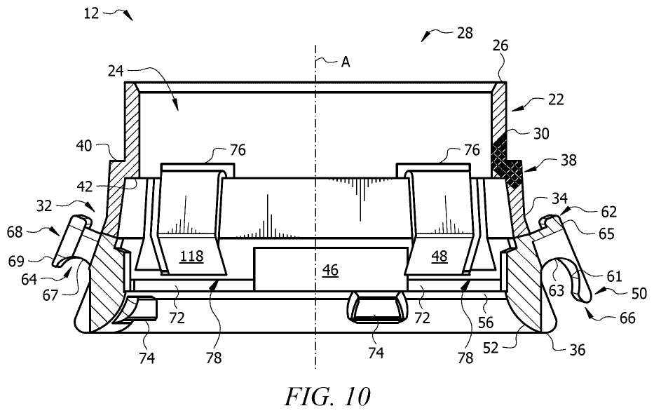

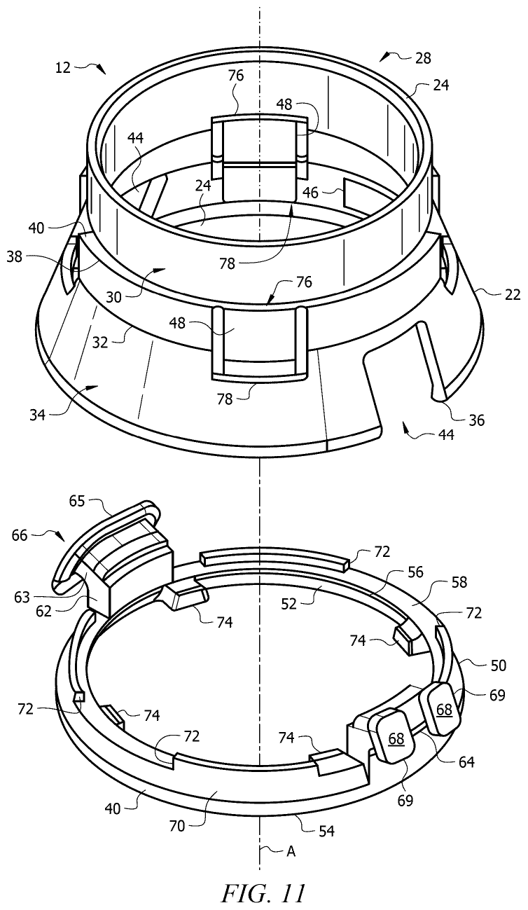

FIG. 10 is a sectional view and FIG. 11 is an exploded, perspective of the collet assembly 12 of the fluid delivery apparatus 10. Referring to FIGS. 9-11, in the exemplary embodiment, the collet assembly 12 includes a collet 22 coupled to a collet lock 50. In the exemplary embodiment, the collet 22 is formed in a generally frustoconical shape, having a hollow interior space 24 defined therein. The collet 22 is formed generally symmetrically about a central axis “A.” An upper rim 26 of the collet 22 defines an opening 28 to the interior space 24. A cylindrical upper wall 30 extends generally vertically downward from the upper rim 26 towards a central portion 32 of the collet 22. A lower wall 34 extends downward at an outward angle from the central portion 32 toward a base 36 (or lower edge) of the collet 22. The upper wall 30, central portion 32, and the lower wall 34 collectively define the interior space 24. A step 38 extends around the upper wall 30, defining an outer horizontal surface 40 (or ledge) configured to engage an attachment band. The step 38 also defines an inner horizontal surface 42 (or step) configured to engage with the plenum assembly 16 to facilitate properly positioning the plenum assembly 16 above a user's skin surface prior to use of the fluid delivery apparatus 10.

As illustrated in FIG. 11, the collet 22 includes a pair of notches, indicated generally at 44, opposite each other and formed through the lower wall 34. In the exemplary embodiment, the notches 44 are generally rectangular in shape and configured to receive a portion of the collet lock 50. In addition, the collet 22 includes one or more stops 46 configured to facilitate positioning of the collet lock 50 when coupled to the collet 22. For example, and without limitation, the one or more stops 46 are formed as inward extending projections formed on lower wall 34. The stops 46 can have form or shape that enables the stops 46 to function as described herein.

As illustrated in FIG. 10 and FIG. 11, in an exemplary embodiment the collet 22 includes a plurality of flexible tabs 48 formed integrally with the upper wall 30. In addition, the plurality of flexible tabs 48 is positioned about and equidistant from the central axis “A.” In particular, the plurality of flexible tabs 48 extends from a first end 76 to an opposite free second end 78. In the exemplary embodiment, the free second end 78 angles radially inward and is configured to engage with the plenum assembly 16 to facilitate properly positioning the plenum assembly 16 at the user's skin surface during use of the fluid delivery apparatus 10.

As illustrated in FIG. 10 and FIG. 11, in the exemplary embodiment, the collet lock 50 is generally ring-shaped, having a convex inner surface 52 extending from a lower outer edge 54 of the collet lock 50 to a generally cylindrical inner wall 56. The inner wall 56 extends upward to an upper surface 58. The collet lock 50 includes a generally cylindrical outer wall 60 that is concentric with inner wall 56 and extends upward from the lower outer edge 54. In addition, the collet lock 50 includes latching members 62, 64, opposite each other and extending upward from the upper surface 58. The latching members 62, 64 are configured to couple to the notches 44 of the collet 22. The latch member 62 includes a first coupling member 66 that extends outward from latch member 62. In particular, the first coupling member 66 includes a neck portion 63 that extends at an upward angle substantially perpendicular to the lower wall 34 of the collet 22. In addition, the first coupling member 66 includes a head portion 65 that extends generally parallel to the lower wall 34 beyond a periphery of the neck portion 63. Furthermore, the first coupling member 66 includes a window or aperture 61 extending through the head portion 65. The window 61 is configured to present an indication to the user of the fluid delivery apparatus 10 of a tightness of the attachment band 430, as is further described herein.

Similarly, the latching member 64 includes an adjacent pair of second coupling members 68 that extend outward from latching member 64. In the exemplary embodiment, the coupling members 68 each include a neck portion 67 that extends at an upward angle substantially perpendicular to the lower wall 34 of the collet 22. In addition, the second coupling members 68 include a head portion 69 that extends generally parallel to the lower wall 34 beyond a periphery of the neck portion 67. The first coupling member 66 and the pair of second coupling members 68 are configured to engage the attachment band 430, as is described further herein.

In the exemplary embodiment, the outer wall 60 of the collet lock 50 includes an upper outer surface 70 that inclines inward at an angle substantially parallel to the lower wall 34 to facilitate face-to-face engagement therewith. In addition, the upper surface 58 includes a plurality of stop members 72 that extend upward and are configured to engage the one or more stops 46 of the collet 22 to facilitate properly positioning of the collet lock 50 when coupled to the collet 22. Extending radially inward from the convex inner surface 52 is a plurality of tabs 74 configured to engage with the plenum assembly 16 to facilitate properly positioning the plenum assembly 16 at the user's skin surface during use of the fluid delivery apparatus 10.

In the exemplary embodiment, the collet 22 is coupled to the collet lock 50 to form a unitary assembly (shown in FIG. 10). In particular, the upper surface 70 and the latching members 62, 64 of the collet lock 50 engage the lower wall 34 and the notches 44 of the collet 22 via a permanent coupling method, for example, and without limitation, via an adhesive bond, a weld joint (e.g., spin welding, ultrasonic welding, laser welding, or heat staking), and the like. Alternatively, the collet 22 and the collet lock 50 may be coupled together using any connection technique that enables the formation of the collet assembly 12.

Additional description of the fluid delivery apparatus 10 seen in FIGS. 7-11, including its operation, can be found, for example, in PCT/US2017/064668 (published as WO 2018/111621 A1), which is hereby incorporated by reference in its entirety.

In some embodiments described herein, medical devices comprising a plurality of microneedles as described herein functions as a permeability enhancer and may increase the delivery of one or more agents through the epidermis. This delivery may occur through modulating transcellular transport mechanisms (e.g., active or passive mechanisms) or through paracellular permeation. Without being bound by any theory, the nanostructured or nanotopography surface may increase the permeability of one or more layers of the viable epidermis, including the epidermal basement membrane by modifying cell/cell tight junctions allowing for paracellular or modifying cellular active transport pathways (e.g., transcellular transport) allowing for diffusion or movement and/or active transport of an administered agent through the viable epidermis and into the underlying viable dermis. This effect may be due to modulation of gene expression of the cell/cell tight junction proteins. As previously mentioned, tight junctions are found within the viable skin and in particular the viable epidermis. The opening of the tight junctions may provide a paracellular route for improved delivery of any agent, such as those that have previously been blocked from delivery through the skin.

Without wishing to be bound by any theory, it is believed that interaction between individual cells and structures of the nanotopography may increase the permeability of an epithelial tissue (e.g., the epidermis) and induce the passage of an agent through a barrier cell and encourage transcellular transport. For instance, interaction with keratinocytes of the viable epidermis may encourage the partitioning of an agent into the keratinocytes (e.g., transcellular transport), followed by diffusion through the cells and across the lipid bilayer again. In addition, interaction of the nanotopography structure and the corneocytes of the stratum corneum may induce changes within the barrier lipids or corneodesmosomes resulting in diffusion of the agent through the stratum corneum into the underlying viable epidermal layers. While an agent may cross a barrier according to paracellular and transcellular routes, the predominant transport path may vary depending upon the nature of the agent.

In some embodiments described herein, the device may interact with one or more components of the epithelial tissue to increase porosity of the tissue making it susceptible to paracellular and/or transcellular transport mechanisms. Epithelial tissue is one of the primary tissue types of the body. Epithelial tissues that may be rendered more porous may include both simple and stratified epithelium, including both keratinized epithelium and transitional epithelium. In addition, epithelial tissue encompassed herein may include any cell types of an epithelial layer including, without limitation, keratinocytes, endothelial cells, lymphatic endothelial cells, squamous cells, columnar cells, cuboidal cells and pseudostratified cells. Any method for measuring porosity may be used including, but not limited to, any epithelial permeability assay. For example, a whole mount permeability assay may be used to measure epithelial (e.g., skin) porosity or barrier function in vivo see, for example, Indra and Leid., Methods Mol Biol. (763) 73-81, which is incorporated by reference herein for its teachings thereof.

In some embodiments described herein, the structural changes induced by the presence of a nanotopography surface on a barrier cell are temporary and reversible. It was surprisingly found that using nanostructured nanotopography surfaces results in a temporary and completely reversible increase in the porosity of epithelial tissues by changing junctional stability and dynamics, which, without being bound by any theory, may result in a temporary increase in the paracellular and transcellular transport of an administered agent through the epidermis and into the viable dermis. Thus, in some aspects, the increase in permeability of the epidermis or an epithelial tissue elicited by the nanotopography, such as promotion of paracellular or transcellular diffusion or movement of one or more agents, returns to a normal physiological state that was present before contacting the epithelial tissue with a nanotopography following the removal of the nanotopography. In this way, the normal barrier function of the barrier cell(s) (e.g., epidermal cell(s)) is restored and no further diffusion or movement of molecules occurs beyond the normal physiological diffusion or movement of molecules within the tissue of a subject.

These reversible structural changes induced by the nanotopography may function to limit secondary skin infections, absorption of harmful toxins, and limit irritation of the dermis. Also, the progressive reversal of epidermal permeability from the top layer of the epidermis to the basal layer may promote the downward movement of one or more agents through the epidermis and into the dermis and prevent back flow or back diffusion of the one or more agents back into the epidermis.

In some embodiments described herein, are methods for applying a device having a plurality of microneedles to the surface of the skin a subject for the treatment of a disease or disorder described herein. In some aspects, the device is applied to an area of the subject's skin, wherein the location of the skin on the body is dense in lymphatic capillaries and/or blood capillaries. Multiple devices may be applied to one or more locations of the skin having a dense network of lymphatic capillaries. In some aspects, 1, 2, 3, 4, 5, or more devices may be applied. These devices may be applied spatially separate or in close proximity or juxtaposed with one another. Exemplary and non-limiting locations dense with lymphatics comprise the palmar surfaces of the hands, the scrotum, the plantar surfaces of the feet and the lower abdomen. The location of the device will be selected based on the medical condition of the patient and the assessment of a medical professional.

In some embodiments described herein, at least a portion of or all of the therapeutic agent may be directly delivered or administered to an initial depth in the skin comprising the nonviable epidermis and/or the viable epidermis. In some aspects, a portion of therapeutic agent may also be directly delivered to the viable dermis in addition to the epidermis. The range of delivery depth will depend on the medical condition being treated and the skin physiology of a given patient. This initial depth of delivery may be defined as a location within the skin, wherein a therapeutic agent first comes into contact as described herein. Without being bound by any theory, it is thought that the administered agent may move (e.g., diffuse) from the initial site of delivery (e.g., the non-viable epidermis, the viable epidermis, the viable dermis, or the interstitium) to a deeper position within the viable skin. For example, a portion of or all of an administered agent may be delivered to the non-viable epidermis and then continue to move (e.g., diffuse) into the viable epidermis and past the basal layer of the viable epidermis and enter into the viable dermis. Alternatively, a portion of or all of an administered agent may be delivered to the viable epidermis (i.e., immediately below the stratum corneum) and then continue to move (e.g., diffuse) past the basal layer of the viable epidermis and enter into the viable dermis. Lastly, a portion of or all of an administered agent may be delivered to the viable dermis. The movement of the one or more active agents throughout the skin is multifactorial and, for example, depends on the liquid carrier composition (e.g., viscosity thereof), rate of administration, delivery structures, etc. This movement through the epidermis and into the dermis may be further defined as a transport phenomenon and quantified by mass transfer rate(s) and/or fluid mechanics (e.g., mass flow rate(s)).

Thus, in some embodiments described herein, the therapeutic agent may be delivered to a depth in the epidermis wherein the therapeutic agent moves past the basal layer of the viable epidermis and into the viable dermis. In some aspects described herein, the therapeutic agent is then absorbed by one or more susceptible lymphatic capillary plexus then delivered to one or more lymph nodes and/or lymph vessels.

In some embodiments described herein, the distribution of depths in the skin, wherein a portion of the one or more agents is initially delivered, which results in uptake of the one or more therapeutic agents by one or more susceptible tumors or inflammatory locus, or by lymph vessels that feed into the tumors or inflammatory locus, ranges from about 5 μm to about 4,500 μm. Because the thickness of the skin can vary from patient to patient based on numerous factors, including, but not limited to, medical condition, diet, gender, age, body mass index, and body part, the required depth to deliver the therapeutic agent will vary. In some aspects, the delivery depth is from about 50 μm to about 4000 μm, from about 100 to about 3500 μm, from about 150 μm to about 3000 μm, from about 200 μm to about 3000 μm, from about 250 μm to about 2000 μm, from about 300 μm to about 1500 μm, or from about 350 μm to about 1000 μm. In some aspects, the delivery depth is about 50 μm, about 100 μm, about 150 μm, about 200 μm, about 250 μm, about 300 μm, about 350 μm, about 400 μm, about 450 μm, about 500 μm, about 600 μm, about 700 μm, about 800 μm, about 900 μm, or about 1000 μm. As used in this context, “about” means ±50 μm.

In some embodiments described herein, the therapeutic agent may be delivered in a liquid carrier solution. In one aspect, the tonicity of the liquid carrier may be hypertonic to the fluids within the blood capillaries or lymphatic capillaries. In another aspect, the tonicity of a liquid carrier solution may be hypotonic to the fluids within the blood capillaries or lymphatic capillaries. In another aspect, the tonicity of a liquid carrier solution may be isotonic to the fluids within the blood capillaries or lymphatic capillaries. The liquid carrier solution may further comprise at least one or more pharmaceutically acceptable excipients, diluent, cosolvent, particulates, or colloids. Pharmaceutically acceptable excipients for use in liquid carrier solutions are known, see, for example, Pharmaceutics: Basic Principles and Application to Pharmacy Practice (Alekha Dash et al. eds., 1st ed. 2013), which is incorporated by reference herein for its teachings thereof.

In some embodiments described herein, the therapeutic agent is present in a liquid carrier as a substantially dissolved solution, a suspension, or a colloidal suspension. Any suitable liquid carrier solution may be utilized that meets at least the United States Pharmacopeia (USP) specifications, and the tonicity of such solutions may be modified as is known, see, for example, Remington: The Science and Practice of Pharmacy (Lloyd V. Allen Jr. ed., 22nd ed. 2012. Exemplary non-limiting liquid carrier solutions may be aqueous, semi-aqueous, or nonaqueous depending on the bioactive agent(s) being administered. For example, an aqueous liquid carrier may comprise water and any one of or a combination of a water-miscible vehicles, ethyl alcohol, liquid (low molecular weight) polyethylene glycol, and the like. Non-aqueous carriers may comprise a fixed oil, such as corn oil, cottonseed oil, peanut oil, or sesame oil, and the like. Suitable liquid carrier solutions may further comprise any one of a preservative, antioxidant, complexation enhancing agent, a buffering agent, an acidifying agent, saline, an electrolyte, a viscosity enhancing agent, a viscosity reducing agent, an alkalizing agent, an antimicrobial agent, an antifungal agent, a solubility enhancing agent or a combination thereof.

In some embodiments described herein, the therapeutic agent is delivered to the viable skin, wherein the distribution of depths in the viable skin for delivery of the agent is immediately past the stratum corneum of the epidermis but above the subcutaneous tissue, which results in uptake of the agent by the lymphatic vasculature of the patient. In some aspects, the depth in the viable skin for delivering one or more agents ranges from about 1 μm to about 4,500 μm beyond the stratum corneum, but still within the viable skin above the subcutaneous tissue.

Non-limiting tests for assessing initial delivery depth in the skin may be invasive (e.g., a biopsy) or non-invasive (e.g., imaging). Conventional non-invasive optical methodologies may be used to assess delivery depth of an agent into the skin including remittance spectroscopy, fluorescence spectroscopy, photothermal spectroscopy, or optical coherence tomography (OCT). Imaging using methods may be conducted in real-time to assess the initial delivery depths. Alternatively, invasive skin biopsies may be taken immediately after administration of an agent, followed by standard histological and staining methodologies to determine delivery depth of an agent. For examples of optical imaging methods useful for determining skin penetration depth of administered agents, see, Sennhenn et al., Skin Pharmacol. 6(2) 152-160 (1993), Gotter et al., Skin Pharmacol. Physiol. 21 156-165 (2008), or Mogensen et al., Semin. Cutan. Med. Surg 28 196-202 (2009), each of which are incorporated by reference herein for their teachings thereof.

In some embodiments described herein are methods for the extended delivery (or administration) of the therapeutic agent as described herein. The medical device comprising a plurality of microneedles is configured such that that the flow rate of the medicament from the device into the patient can be adjusted. As such, the length of time required will vary accordingly. In some aspects, the flow rate of the medical device is adjusted such that the medicament is administered over from about 0.5 hours to about 72 hours. In some aspects the time period for administration is about 1 hour, 2 hours, 3 hours, 4 hours, 5 hours, 6 hours, 7 hours, 8 hours, 9 hours, 10 hours, 12 hours, 15 hours 18 hours, 21 hours, 24 hours, 27 hours, 30 hours, 33 hours, 36 hours, 39 hours, 42 hours, 45 hours, 48 hours, 51 hours, 54 hours, 57 hours, 60 hours, 63 hours, 66 hours, 69 hours or 72 hours. In other aspects, the time period for administration is selected based on the medical condition of the patient and an assessment by the medical professional treating the patient.

In some embodiments described herein, one or more agents in a liquid carrier solution are administered to an initial approximate volume of space below the outer surface of the skin. The one or more therapeutic agents in a liquid carrier solution initially delivered to the skin (e.g., prior to any subsequent movement or diffusion) may be distributed within, or encompassed by an approximate three dimensional volume of the skin. The one or more initially delivered agents exhibits a Gaussian distribution of delivery depths and will also have a Gaussian distribution within a three dimensional volume of the skin tissue. In some embodiments described herein, the flow rate of the therapeutic agent to the skin per single microneedle as described herein may be about 0.01 μl per hour to about 500 μl per hour. In some aspects, the flow rate for each individual microneedle is from about 0.1 μl per hour to about 450 μl per hour, about 0.5 μl per hour to about 400 μl per hour, about 1.0 μl per hour to about 350 μl per hour, about 5.0 μl per hour to about 300 μl per hour, about 5.0 μl per hour to about 250 μl per hour, about 10 μl per hour to about 200 μl per hour, about 15 μl per hour to about 100 μl per hour, or about 20 μl per hour to about 50 μl per hour. In some aspects, the flow rate for each individual microneedle is about 1 μl per hour, 2 μl per hour, 5 μl per hour, 10 μl per hour, 15 μl per hour, 20 μl per hour, 25 μl per hour, 30 μl per hour, 40 μl per hour, 50 μl per hour, 75 μl per hour, or 100 μl per hour. Each individual microneedle will have a flow rate that contributes to the overall device flow rated. The maximum overall flow rate will be flow rate of each individual microneedle multiplied by the total number of microneedles. The overall controlled flow rate of all of the combined microneedles may be from about 0.2 μl per hour to about 50,000 μl per hour. The medical device is configured such that that the flow rate can be controlled appropriately. The flow rate will be based upon the medical condition of the patient and an assessment by the medical professional treating the patient.

### Additional Embodiments

Additional non-limiting and non-exhaustive embodiments (referred to as “Additional Embodiment” or “additional Embodiments”) are provided below.

Additional Embodiment 1. A method of treating a subject having, or suspected of having, an arthritic disease or associated condition, or a symptom associated with an arthritic disease or associated condition, by administering a therapeutically effective amount of a therapeutic agent to the lymphatic system of the subject, the method comprising: placing a first medical device comprising a plurality of microneedles on the skin of the subject at a first location proximate to a first position under the skin of the subject, wherein the first position is proximate to lymph vessels and/or lymph capillaries that drain into a lymphatic duct, and wherein the microneedles of the first medical device have a surface comprising nanotopography; inserting the plurality of microneedles of the first medical device into the subject to a depth whereby at least the epidermis is penetrated and an end of at least one of the microneedles is proximate to the first position; and administering via the microneedles of the first medical device a first dose of the anti-inflammatory agent into the first position; thereby delivering the therapeutically effective amount of the therapeutic agent to the lymphatic system of the subject.

Additional Embodiment 2. A method of increasing lymphatic amount or lymphatic concentration of a therapeutic agent in the lymphatic system of a subject having, or suspected of having, an arthritic disease or associated condition, or a symptom associated with an arthritic disease or associated condition, the method comprising: placing a first medical device comprising a plurality of microneedles on the skin of the subject at a first location proximate to a first position under the skin of the subject, wherein the first position is proximate to lymph vessels and/or lymph capillaries that drain into a lymphatic duct, and wherein the microneedles of the first medical device have a surface comprising nanotopography; inserting the plurality of microneedles of the first medical device into the subject to a depth whereby at least the epidermis is penetrated and an end of at least one of the microneedles is proximate to the first position; and administering via the microneedles of the first medical device a first dose of the anti-inflammatory agent into the first position; thereby increasing the lymphatic concentration of the therapeutic agent in the lymphatic system of the subject.

Additional Embodiment 3. A method of decreasing an elevated lymphatic amount or lymphatic concentration of an inflammatory substance in a subject having, or suspected of having, an arthritic disease or associated condition, or a symptom associated with an arthritic disease or associated condition, wherein the elevated lymphatic amount or lymphatic concentration results from the presence of the disease or associated condition, the method comprising: placing a first medical device comprising a plurality of microneedles on the skin of the subject at a first location proximate to a first position under the skin of the subject, wherein the first position is proximate to lymph vessels and/or lymph capillaries that drain into a lymphatic duct, and wherein the microneedles of the first medical device have a surface comprising nanotopography; inserting the plurality of microneedles of the first medical device into the subject to a depth whereby at least the epidermis is penetrated and an end of at least one of the microneedles is proximate to the first position; and administering via the microneedles of the first medical device a therapeutically effective amount of the therapeutic agent into the first position; wherein the therapeutically effective amount comprises an amount that results in a reduction in the lymphatic amount or lymphatic concentration of the inflammatory substance in the lymphatic system of the subject.

Additional Embodiment 4. A method of increasing lymphatic pumping rate of at least on lymph node in a subject having, or suspected of having, an arthritic disease or associated condition, or a symptom associated with an arthritic disease or associated condition, the method comprising: placing a first medical device comprising a plurality of microneedles on the skin of the subject at a first location proximate to a first position under the skin of the subject, wherein the first position is proximate to lymph vessels and/or lymph capillaries that drain into a lymphatic duct, and wherein the microneedles of the first medical device have a surface comprising nanotopography; inserting the plurality of microneedles of the first medical device into the subject to a depth whereby at least the epidermis is penetrated and an end of at least one of the microneedles is proximate to the first position; and administering via the microneedles of the first medical device a therapeutically effective amount of the therapeutic agent into the first position; wherein the therapeutically effective amount comprises an amount that results in an increased lymphatic pumping rate of the at least on lymph node in the lymphatic system of the subject.

Additional Embodiment 5. A method of achieving or restoring a normal pumping rate of at least one lymph node in a subject having, or suspected of having, an arthritic disease or associated condition, or a symptom associated with an arthritic disease or associated condition, wherein the pumping rate is reduced as a result of the arthritic disease or associated condition, the method comprising: placing a first medical device comprising a plurality of microneedles on the skin of the subject at a first location proximate to a first position under the skin of the subject, wherein the first position is proximate to lymph vessels and/or lymph capillaries that drain into a lymphatic duct, and wherein the microneedles of the first medical device have a surface comprising nanotopography; inserting the plurality of microneedles of the first medical device into the subject to a depth whereby at least the epidermis is penetrated and an end of at least one of the microneedles is proximate to the first position; and administering via the microneedles of the first medical device a therapeutically effective amount of the therapeutic agent into the first position; wherein the therapeutically effective amount comprises an amount that results in restoration of the normal pumping rate to a rate that is comparable to or greater than the pumping rate in a subject that does not have the disease or associated condition.

Additional Embodiment 6. The method of any of Additional Embodiments 1-5, wherein the therapeutically effective amount comprises: an amount or concentration that is effective to treat the disease or associated condition; or an amount or concentration that is effective to reduce or eliminate at least one symptom or clinical manifestation of the disease or associated condition.

Additional Embodiment 7. The method of any of Additional Embodiments 1-6, wherein the arthritic disease or associated condition is selected from the group consisting of: rheumatoid arthritis (RA); juvenile arthritis; psoriatic arthritis; ankylosing spondylitis; gout; and combinations thereof.

Additional Embodiment 8. The method of any of Additional Embodiments 1-7, wherein the associated condition comprises another autoimmune condition.

Additional Embodiment 9. The method of any of Additional Embodiments 1-8, wherein the associated condition comprises an autoimmune condition selected from the group consisting of scleroderma, lupus ulcerative colitis (UC), Crohn's disease, plaque psoriasis, autoimmune uveitis, Behcet's disease, and sarcoidosis.

Additional Embodiment 10. The method according to any of Additional Embodiments 1-9, wherein the therapeutic agent comprises an anti-inflammatory agent.

Additional Embodiment 11. The method according to any of Additional Embodiments 1-10, wherein the therapeutic agent comprises an anti-arthritic agent.

Additional Embodiment 12. The method according to any of Additional Embodiments 1-11, wherein the therapeutic agent comprises an agent that reduces TNFa activity.

Additional Embodiment 13. The method according to any of Additional Embodiments 1-12, wherein the therapeutic agent is selected from the group consisting of: Adalimumab (Humira®); Adalimumab-atto (Amjevita®); Certolizumab pegol (Cimzia®); etanercept (Enbrel®); etanercept-szzs (Ereizi®); Golimumab (Simponi®, Simponi Aria®); Infliximab (Remicade®); Infliximab-dyyb (Inflectra®); analogs thereof; variants thereof; biosimilars thereof; bioequivalents thereof; and combinations thereof.

Additional Embodiment 14. The method of any of Additional Embodiments 1-13, wherein the therapeutically effective amount of the therapeutic agent comprises a dose-sparing amount of the therapeutic agent.

Additional Embodiment 15. The method of any of Additional Embodiments 1-14, wherein the therapeutically effective amount of the therapeutic agent is effective to reduce a DAS28 (ESR) and/or a DAS28(CRT) score by at least 10%, at least 20%, at least 30%, at least 40%, at least 50%, at least 60%, at least 70%, or greater compared to a DAS28 (ESR) and/or a DAS28(CRT) determined in the subject prior to administering the therapeutic agent.

Additional Embodiment 16. The method of any of Additional Embodiments 1-15, wherein the therapeutically effective amount of the therapeutic agent is effective to reduce a 66/68-joint count and/or a 28-joint count score by at least 10%, at least 20%, at least 30%, at least 40%, at least 50%, at least 60%, at least 70%, or greater compared to a 66/68-joint count and/or a 28-joint count determined in the subject prior to administering the therapeutic agent.

Additional Embodiment 17. The method of any of Additional Embodiments 1-16, wherein the therapeutically effective amount of the therapeutic agent is effective to reduce patient rating of overall disease activity by at least 10%, at least 20%, at least 30%, at least 40%, at least 50%, at least 60%, at least 70%, or greater compared to a patient rating of overall disease activity determined in the subject prior to administering the therapeutic agent.

Additional Embodiment 18. The method of any of Additional Embodiments 1-17, wherein the therapeutically effective amount of the therapeutic agent is effective to improve ACR response by at least 10%, at least 20%, at least 30%, at least 40%, at least 50%, at least 60%, at least 70%, or greater compared to a an ACR response determined in the subject prior to administering the therapeutic agent.

Additional Embodiment 19. The method according to any one of Additional Embodiments 1-18, wherein the subject is a mammal.

Additional Embodiment 20. The method according to any one of Additional Embodiments 1-19, wherein the subject is a human.

Additional Embodiment 21. The method according to any one of Additional Embodiments 1-20, wherein the medical device is a Sofusa™ Lymphatic Delivery System (SOFUSA).

Additional Embodiment 22. The method according to any one of Additional Embodiments 1-21, wherein the medical device comprises a fluid delivery apparatus, wherein the fluid delivery apparatus comprises: a fluid distribution assembly wherein a cap assembly is coupled to a cartridge assembly, and the cartridge assembly is slidably coupled to a plenum assembly, and a mechanical controller assembly is slidably coupled to the cartridge assembly; a collet assembly constituting the housing of the fluid delivery apparatus and being slidably coupled to the fluid distribution assembly; and a plurality of microneedles fluidically connected with the fluid distribution assembly having a surface comprising nanotopography, the plurality of microneedles being capable of penetrating the stratum corneum of the skin of a subject and controllably delivering the therapeutic agent to a depth below the surface of the skin.

Additional Embodiment 23. The method according to any one of Additional Embodiments 1-22, wherein the medical device delivers the therapeutic agent to a depth below the surface of the skin of from about 50 μm to about 4000 μm, from about 250 μm to about 2000 μm, or from about 350 μm to about 1000 μm.

Additional Embodiment 24. The method according to any one of Additional Embodiments 1-23, wherein each of the microneedles in the medical device has a length between about 200 to about 800 μm, between about 250 to about 750 μm, or between about 300 to about 600 μm.

Additional Embodiment 25. The method according to any one of Additional Embodiments 1-24, wherein the therapeutic agent comprises Enbrel.

Additional Embodiment 26. A dose sparing amount or concentration of a therapeutic agent that is therapeutically effective for treating an arthritic disease or associated condition, or a symptom or associated with an arthritic disease or associated condition, upon administration via a medical device that administers the dose-sparing amount or concentration to the lymphatic system of a subject having, or suspected of having, the arthritic disease or associated condition.

### EXAMPLES

### Example 1—Exemplary Sofusa Lymphatic Delivery System

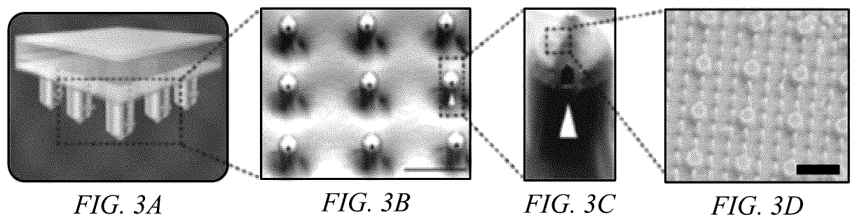

An exemplary Sofusa Lymphatic Delivery System (SOFUSA) was designed to access the lymphatic system directly through the skin at the epidermal/dermal boundary (See FIG. 1) such that the infusion accessed afferent lymphatic capillaries when employed as described in Examples 2 and 3, below. The exemplary SOFUSA system was comprised of a microneedle device (see, e.g., FIG. 3A) and a syringe pump configured to administer anti-arthritic therapeutic agent, such as Enbrel, into the lymphatic circulation through the skin. This configuration was optimized to provide flow rates and pharmacokinetics using lymphatic anti-arthritic drug delivery in early phase clinical trials. The exemplary SOFUSA device was attached to the body of subjects via an attachment system that domed the skin upwards for consistent microneedle penetration, and a nylon strap with Velcro® that comfortably held the device down on the skin, keeping the microneedles in position until the dosing was complete (See, e.g., FIG. 2). Dose volume and infusion rate were set with an infusion pump (See, e.g., FIG. 2).

The exemplary SOFUSA microneedles included a nanotopographical imprinted polymer film heat-formed over each microneedle on the array (See, e.g., FIGS. 3B-3D). The microneedles contained a through-channel to facilitate drug delivery (See, e.g., FIG. 3C). Without wishing to be bound by any theory, the nanotopographical film-microneedle combination is believed to increase permeability through the skin epidermis layer by remodeling tight junction proteins initiated via integrin binding to the nanotopography. Additionally, nanotopography draped microneedles have been demonstrated to result in a 10-fold increase in serum concentration of administered agents vs. undraped microneedles (See, e.g., Walsh, L. et al., Nano Lett. 15, 2434-2441 (2015)). Without wishing to be bound by any theory, the increased permeability is in turn believed to enable the exemplary SOFUSA device to administer therapeutically effective agent amounts or concentration, and control targeting of such administration to the lymphatic system, based on the administration of agents between the stratum corneum and initial lymphatic capillaries.

SOFUSA microneedles were arranged into an array and placed on the skin using a mechanical impact applicator. A solution comprising Enbrel at a concentration of approximately 50 milligrams per milliliter (50 mg/mL) was then administered at a controlled infusion rate into the device, through the microneedles and into the skin using an external syringe pump. The solution comprising Enbrel was delivered into the body at an infusion rate of 0.5 milliliters per hour (mL/hr), which corresponded to an infusion period of one hour to deliver a 25 mg dose and 2 hours to deliver a 50 mg dose.

### Example 2—Study Design

A Phase 1b proof-of-concept, open-label study is being conducted to assess the safety and pilot efficacy of Enbrel administered by the SOFUSA system for the treatment of patients with moderately to severely active Rheumatoid Arthritis (RA) and who have demonstrated an inadequate response to weekly subcutaneous administration of 50 mg of Enbrel. In this ongoing Phase 1b study, 25 mg of Enbrel (50% of the non-SOFUSA delivered subcutaneous dose) was administered lymphatically once weekly via SOFUSA to patients for 12 weeks. The study design calls for an increase in the SOFUSA-administered Enbrel dose to 50 mg during the dose escalation phase of the study (weeks 4-8) provided that dose escalation criteria are met. Patients remain on either the 25 mg or 50 mg dose for the final maintenance phase of the study. The dose escalation criteria for the Enbrel dose to be increased from 25 mg to 50 mg are: a DAS28 (ESR)≥3.2 and an increase in DAS28 (ESR)>0.6 compared to baseline.

The patients evaluated in the study are/were those who demonstrated an inadequate response after at least 3 months of Enbrel subcutaneous therapy as evidenced by a disease activity level that remains moderate to severe. Patients entered in the study had been treated with subcutaneous Enbrel with or without continuation of MTX, and have had either a primary (lack of sufficient efficacy with initial treatment) or secondary (initial efficacy but became inefficacious over time) inadequate response with the maximum approved dose of Enbrel. However, these patients were differentiated from non-responders who showed no improvement in RA disease measures due to Enbrel therapy and therefore were excluded from the study. To the extent that inadequate response to Enbrel in some patients may be due to insufficient Enbrel levels in target lymphoid tissues or an inadequate immunologic response, Enbrel administered into the lymphatic system via SOFUSA was therefore theorized to provide a favorable clinical response despite an inadequate response to subcutaneous Enbrel administration.

### Example 3—Case Presentations

A 43-year-old female patient (ID 01-002) presented with an inadequate response to Enbrel after 11 months of once weekly 50 mg Enbrel subcutaneous injections. The patient was diagnosed with RA approximately 2 years earlier. The patient was also on once weekly methotrexate subcutaneous injections of 25 mg for 20 months, and oral once daily prednisone at 5 mg for 5 months. The patient weighed 278 pounds, corresponding to a BMI of 52.5 kg/m². The patient had a score of 7 utilizing the ACR/EULAR (2010) classification criteria (See, e.g., Aletaha, D. et al., Arthritis Rheum., 62, 2569-2581 (2010)) and was classified as Class III on the ACR 1991 global functioning status (See, e.g., Hochberg, M. C. et al., Arthritis Rheum., 35, 498-502 (1992)) and being able to perform usual self-care activities but limited in vocational and avocational activities. The patient had a complex medical history which included type 2 diabetes, obesity, essential hypertension, asthma, coronary artery disease, Raynaud's syndrome, venous stasis, hyperlipidemia, depression, gastroesophageal reflux disease, irritable bowel syndrome, nephrectomy for renal cell carcinoma, and renal insufficiency along with associated medications, which included albuterol and fluticasone inhaler, clopidogrel, diltiazem, duloxetine, furosemide, metoprolol, nitroglycerine, rosuvastatin, and valsartan.

This patient received SOFUSA-administered Enbrel at a 25 mg dosage once weekly for 12 weeks. Each week, SOFUSA was applied to the dorsal forearm with alternating left and right arm locations and the SOFUSA-administered Enbrel solution was infused through the skin into the lymphatic circulation for one hour.

### Patient 01-004

A 69-year-old male Caucasian patient (ID 01-004) presented with an inadequate response to Enbrel after 15 months of once weekly 50 mg Enbrel SC injections. At screening, the patient weighed 220 lbs with a height of 6′ 3″ corresponding to a BMI of 27.5 kg/m². The patient was diagnosed with Rheumatoid Arthritis (RA) approximately 6 years earlier. The patient was also on hydroxychloroquine 200 mg PO bid for 6 months and acetaminophen 500 mg×2 PO bid for 6 years. Prior to beginning Enbrel therapy, the patient had been on sulfasalazine 500 mg×2 pills BID, and then moved to prednisone at 10 mg×2 pills PO BID, and then moved to methotrexate at 20 mg/0.4 mL SC QW.

Patient ID 01-004 had a score of 6 on the ACR/EULAR (2010) classification criteria (Aletaha 2010) and was classified as Class III on the ACR 1991 global functioning status (Hochberg, 1992) being able to perform usual self-care activities but limited in vocational and avocational activities. The patient had a complex medical history which includes type 2 diabetes, diabetic neuropathy, hypothyroidism, GERD, back pain, herniated cervical/lumbar discs, spinal laminectomy, cervical spondylosis, lumbar fusion, back nerve ablation, hernia repair, Schaumburg's Disease. Additional medications include insulin glargine, insulin aspart, metformin and sitagliptin for type 2 diabetes, gabapentin for back pain, terazosin for benign hyperplasia, famotidine for GERD, and levothyroxine for hypothyroidism.

As with Patient ID 01-002, this patient ID 01-004 received SOFUSA-administered Enbrel at a 25 mg dosage once weekly for 12 weeks. Each week, SOFUSA was applied to the dorsal forearm with alternating left and right arm locations and the SOFUSA-administered Enbrel solution was infused through the skin into the lymphatic circulation for one hour.

### Patient 01-006

A 51-year-old female patient (ID 01-006) presented with an inadequate response to Enbrel after 2 years of once weekly 50 mg Enbrel SC injections. At Screening, the patient weighed 266 lbs with a height of 5′ 5″ corresponding to a BMI of 44.3 kg/m2. The patient was diagnosed with RA approximately 2 years earlier. The patient was also on methotrexate 20 mg PO qd and folic acid 1 mg PO qd both for 2.5 years while on Enbrel. Prior to beginning Enbrel therapy, the patient had been on methotrexate 12.5 mg PO QW, and was also placed on prednisone 5 mg PO QD one month after beginning Enbrel therapy. One month after adding prednisone at 5 mg PO QD, the prednisone dosage increased to 10 mg PO QD.

Patient had a score of 9 on the ACR/EULAR (2010) classification criteria (Aletaha 2010) and was classified as Class I on the ACR 1991 global functioning status (Hochberg, 1992) being completely able to perform usual activities of daily living (self-care, vocational, avocational). Patient has a medical history which includes rheumatoid arthritis, osteoarthritis, inflammatory polyarthritis, and venous reflux disease in both legs. Additional medications include naproxen and ibuprofen for osteoarthritis pain.

As with Patient ID 01-00 and Patient ID 01-004, this patient ID 01-006 received SOFUSA-administered Enbrel at a 25 mg dosage once weekly for 12 weeks. Each week, SOFUSA was applied to the dorsal forearm with alternating left and right arm locations and the SOFUSA-administered Enbrel solution was infused through the skin into the lymphatic circulation for one hour.

### DAS28 (ESR/CRP) Results

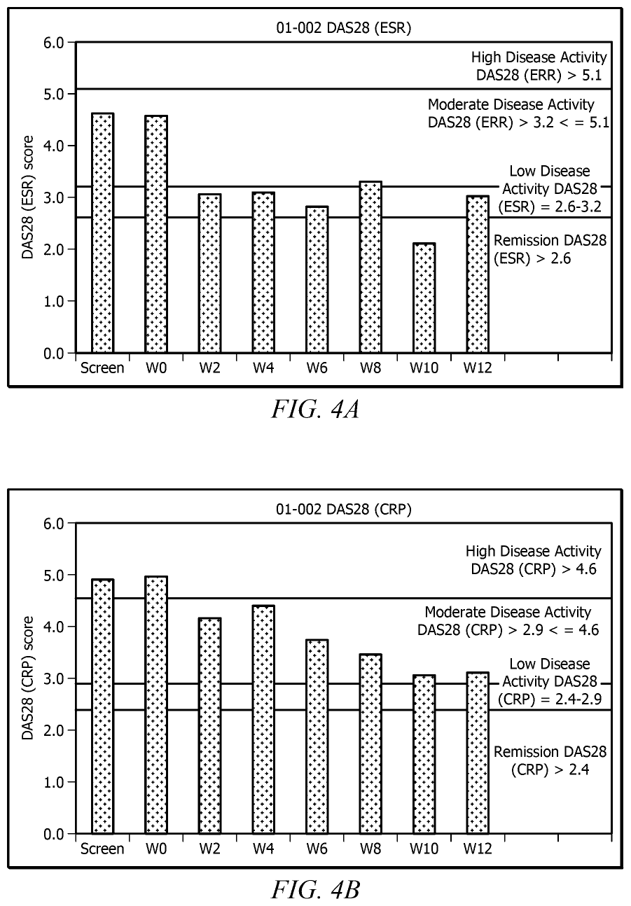

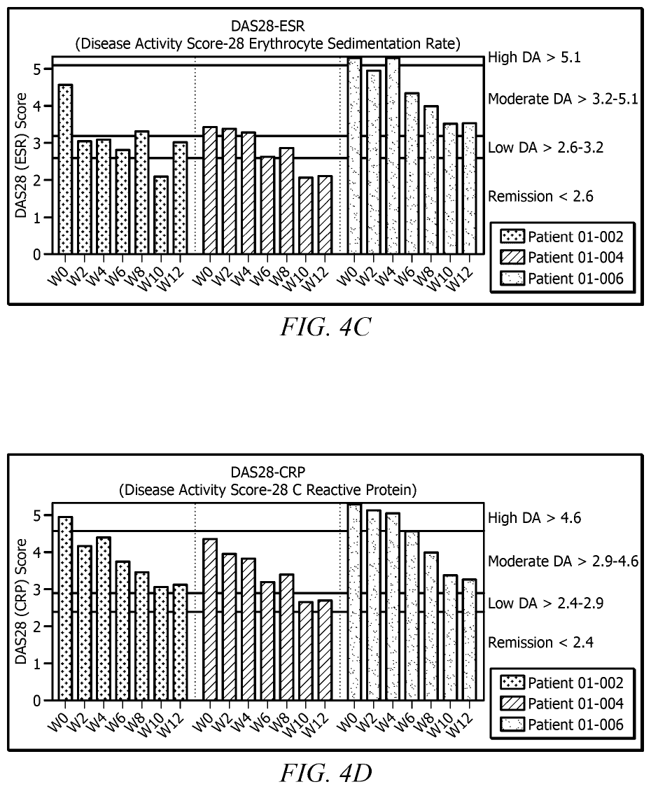

Disease Activity Scores (DAS) 28 (DAS28) DAS28) were calculated based on 28 tender and swollen joints, Patient Global Assessment of Disease Activity, and C-Reactive Protein (CRP) or Erythrocyte Sedimentation Rate (ESR). DAS28 (ESR) and DAS28 (CRP) scores for Patient ID 01-002 are shown in FIG. 4A and FIG. 4B, respectively. As indicated in FIG. 4A, DAS28 (ESR) at screening and baseline was 4.62 and 4.58, respectively, indicating moderate disease activity. As indicated in FIG. 4B, DAS28 (CRP) at screening and baseline was 4.94 and 4.99 respectively, indicating high disease activity.

As indicated in FIG. 4A and FIG. 4B, the DAS28 scores for patient IS 0-002 declined over time and remained low compared to baseline throughout the study. As a result, the patient did not meet the dose escalation criteria during the dose escalation period and remained on 25 mg for the full 12 weeks.

After 12 weeks, DAS28 (ESR) decreased 34.1% from 4.58 at baseline to 3.02 at week 12, demonstrating a change from moderate disease activity to low disease activity (See FIG. 4A). Similarly, DAS28 (CRP) decreased 37.5% from 4.99 at baseline to 3.12 at Week 12 demonstrating a change from high disease activity to moderate disease activity (See FIG. 4B). The lowest DAS28 (ESR) achieved was 2.10 at Week 10 after 10 weekly doses, which corresponds to a disease activity level of remission. An open label extension study has been IRB-approved to evaluate potential for further dose reductions in patients who respond well at 25 mg of weekly SOFUSA-administered Enbrel dosing.

The DAS28-ESR and DAS28-CRP results for Patient ID 01-004 and Patient ID 01-006 are depicted alongside the results for Patient ID 01-002 in FIG. 4C and FIG. 4D, respectively.

### Tender and Swollen Joint Counts

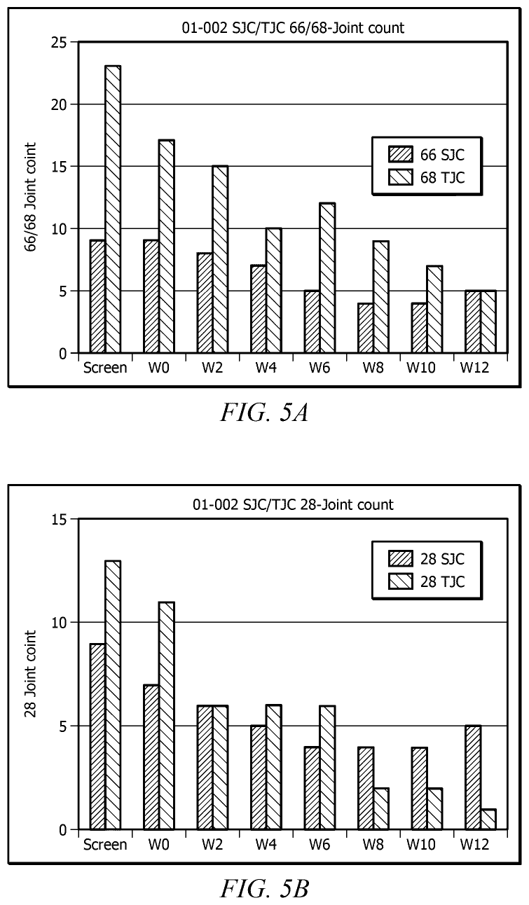

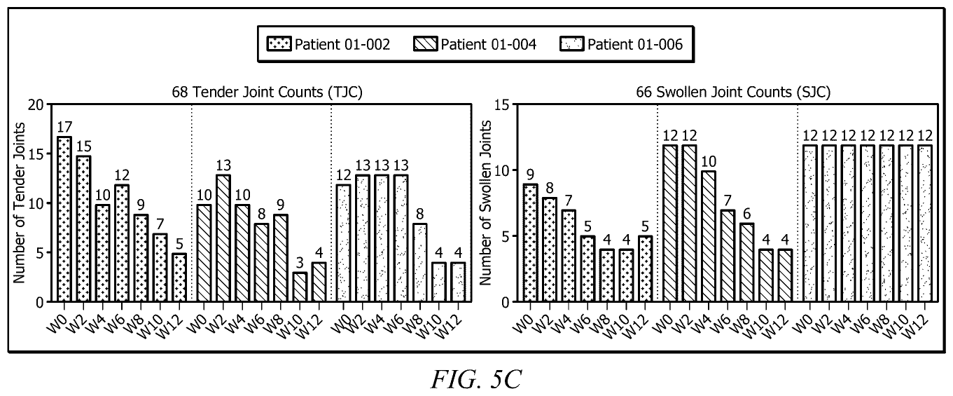

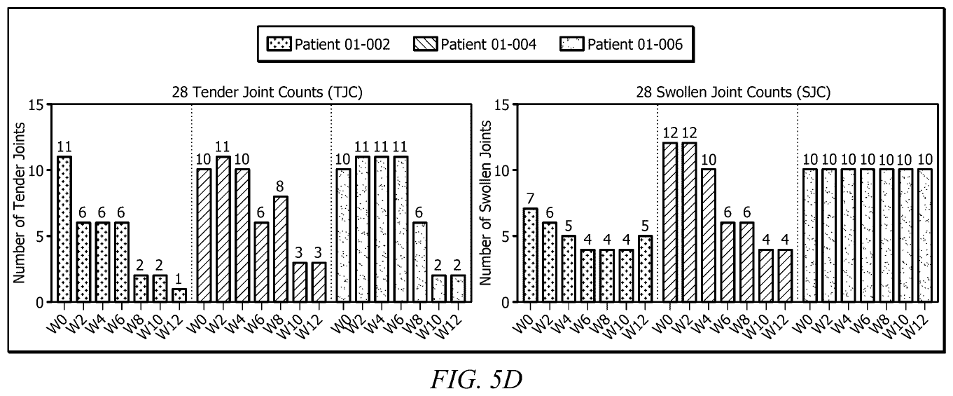

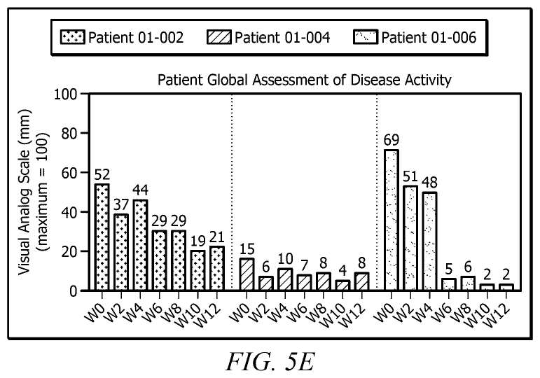

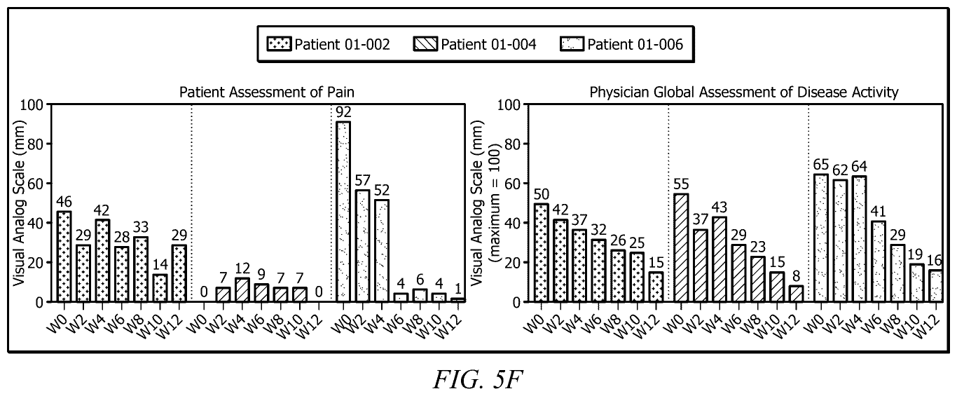

Joint counts were performed every 2 weeks. There was observed a consistent decrease in the number of tender and swollen joints using both 66/68 and 28 joint count criteria for the entire 12-week dosing period (See FIG. 5A and FIG. 5B, respectively). In particular, the number of tender joints decreased from week 0 to week 12 by 70.6% (full 68-Joint Count) and 90.9% (28 Joint Count). Similarly, the number of swollen joints decreased from week 0 to week 12 by 44.4% (full 66-Joint Count) and 28.6% (28-Joint Count).

The 66 joint count, 68 joint count, and 28 joint count results for Patient ID 01-004 and Patient ID 01-006 are depicted alongside the results for Patient ID 01-002 in FIG. 5C and FIG. 5D, respectively.

### Patient/Physician Disease Activity/Pain Visual Analog Scores

Ratings were performed by each patient and physician on a Visual Analog Scale (VAS) every 2 weeks for disease activity (patient and physician) and disease-related pain (patient). Patient ID 01-002 rating of overall disease activity (Patient Global Assessment of Disease Activity) on a Visual Analog Scale (100 mm; horizontal) declined from 52 mm at week 0 to 21 mm at week 12, corresponding to a 59.6% overall reduction in disease activity. In addition, the Patient Assessment of Pain for Patient ID 01-002 decreased from 46 mm at week 0 to 29 mm at week 12 (i.e., 37.0% decrease). The Physician Global Assessment of Disease Activity for Patient ID 01-002 decreased substantially from 50 mm at week 0 to 15 mm at week 12, corresponding to a 70.0% decrease in disease activity.

The Patient Global Assessment of Disease Activity for Patient ID 01-004 and Patient ID 01-006 are depicted alongside the results for Patient ID 01-002 in FIG. 5E. The Patient Assessment of Pain and Physician Global Assessment of Disease Activity scores for Patient ID 01-004 and Patient ID 01-006 are depicted alongside the results for Patient ID 01-002 in FIG. 5F

### ACR Response

An ACR 50% response was achieved at week 10 as defined by ACR response criteria (See, e.g., Felson, D. T. et al., Arthritis Rheum. 36, 729-740 (1993)), coincident with a DAS28 (ESR) of 2.10 for Patient 01-002 and Patient 01-004, and indicative of remission (see, e.g., FIG. 4C). The ACR 50% response was achieved by demonstrating at least a 50% improvement in TJC68, SJC66, and Patient/Physician VAS assessments compared to baseline (week 0).

As depicted in FIG. 5C, FIG. 5E, and FIG. 5F, for Patient 01-002, TJC68 decreased 58.8%, SJC66 decreased 55.6%, Patient Global Assessment of Disease Activity (VAS) decreased 63.5%, Patient Assessment of Pain (VAS) decreased 69.6%, and Physician Global Assessment of Disease Activity (VAS) decreased 50.0% by week 12 of the study. As depicted in FIG. 5C, FIG. 5E, and FIG. 5F, for Patient ID 01-004, TJC68 decreased 60.0%, SJC66 decreased 66.7%, Patient Global Assessment of Disease Activity (VAS) decreased 46.7%, Patient Assessment of Pain (VAS) decreased 100.0%, and Physician Global Assessment of Disease Activity (VAS) decreased 85.4% (however, Patient Global Assessment of Disease Activity (VAS) had decreased by 73.3% at week 10 of the study). As depicted in FIG. 5C, FIG. 5E, and FIG. 5F, for Patient ID 01-006, TJC68 decreased 70.6%, SJC66 was unchanged, Patient Global Assessment of Disease Activity (VAS) decreased 97.1%, Patient Assessment of Pain (VAS) decreased 98.9%, and Physician Global Assessment of Disease Activity (VAS) decreased 75.4%.

### Lymphatic Imaging

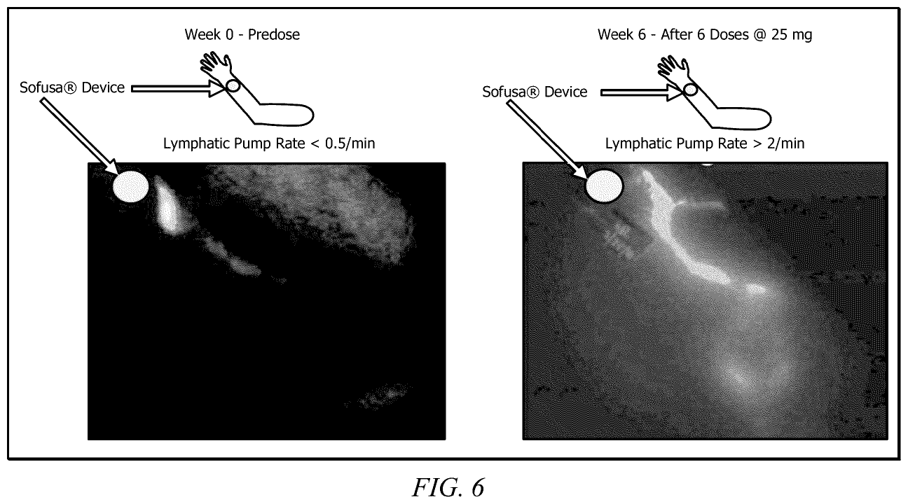

The local lymphatic function was measured using Near-Infrared Fluorescence (NIRF) imaging techniques with Indocyanine Green (ICG) at the beginning, at 6 weeks, and at the end of the study. As illustrated in FIG. 6, which contain video images from week 0 before SOFUSA treatment and after 6 Enbrel doses were administered using SOFUSA. The ICG was delivered at the opposite arm location as the Enbrel dosing. As indicted in FIG. 6, at week 0, very few lymphatic vessels were visible suggesting low lymphatic function, and the pump rate was counted at less than 0.5 pumps per minute. At week 6, after 6 SOFUSA Enbrel doses, significantly more lymphatic vessels could be imaged, and the pump rate was observed to increase to greater than 2 pumps per minute.

The reasons for primary and secondary treatment failure in individual patients suffering from RA are often multifactorial and historically have not been well understood. As demonstrated in the Examples above, Enbrel administered into the lymphatic system via SOFUSA resulted in a favorable clinical response despite previous inadequate response in patients to subcutaneous Enbrel administration, consistent with the believe that that inadequate response to conventional (i.e., non-lymphatic) administration of Enbrel in patients may be due to insufficient Enbrel levels in target lymphoid tissues or an inadequate immunologic response.

In this regard, it has been speculated that lymphatic dysfunction may encourage the development of rheumatic autoimmune diseases such as scleroderma, lupus, and rheumatoid arthritis (see, e.g., Aletaha, D., et al., Ann. Rheum. Dis., 75, 1479-1485 (2016)). Given the importance of the lymphatic system in autoimmune conditions, imaging of the lymphatics was conducted in the studies described above to demonstrate that Enbrel administered via SOFUSA is delivered into lymphatic vessels, and to determine if lymphatic flow and pumping improved in patients receiving SOFUSA with Enbrel. NIRF imaging of the lymphatics after infusion of a solution of ICG at the beginning, middle and end of the study. NIRF imaging was used to assess lymphatic pumping in RA patients before and after treatment with SOFUSA with Enbrel. Additionally, the imaging provided a visualization of the lymphatic vessels and was intended to confirm that SOFUSA delivers ICG solution directly into the lymphatics.

### Example 4—Etanercept Lymph Node Concentrations

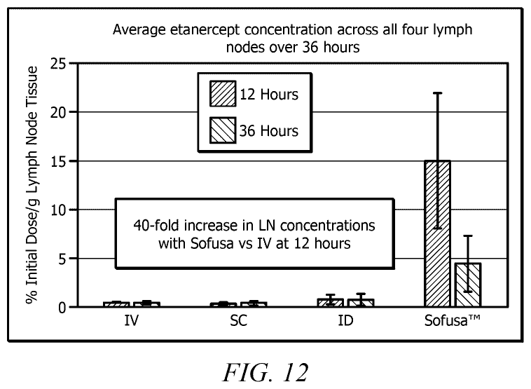

Etanercept was administered to subjects either via intravenous (IV), subcutaneous (SC), Intradermal (ID), or lymphatic (Sofusa) delivery. Average etanercept concentrations (expressed as percentage initial dose per gram of lymph tissue) in lymph node tissue was then determined at twelve and 36 hours post-administration. As depicted in FIG. 12, lymphatic-mediated delivery results in far greater, and superior concentrations of etanercept in lymph nodes compared to all other tested delivery routes. In particular, etanercept concentrations at 12 hours post lymphatic (Sofusa) administration were approximately 40-fold greater, and approximately 9-fold greater at 36 hours, than etanercept concentrations administered via IV.

As demonstrated in the Examples above, lymphatic administration, such as via SOFUSA, of as little as half the dose of a typical subcutaneous dose of an anti-inflammatory agent, such as Enbrel, resulted in profound improvement in all measured indicia of efficacious arthritic therapy in subjects who were demonstrably poorly responsive, non-responsive or refractory to conventional Enbrel therapy and administration route (e.g., subcutaneous injection). Such SOFUSA-mediated lymphatic administration concomitantly resulted in improved lymphatic flow and pumping rate. Hence, SOFUSA-mediated lymphatic delivery or administration of anti-inflammatory agents provides not only for enhanced therapeutic benefit in subjects having, or suspected of having an arthritic disease or associated condition, or one or more symptoms or clinical manifestations thereof, but provides for such benefit by administering a significantly lower dose relative to non-lymphatic administration or delivery routes. Accordingly, such SOFUSA-mediated administration surprisingly and advantageously provides for dose-sparing administration of therapeutically effective amounts or concentrations of anti-inflammatory agent for treating such arthritic diseases or associated conditions, and/or for reducing one or more symptoms or clinical manifestations thereof. Without wishing to be bound by any theory, such benefits appear to result from increase amount and/or concentration of the anti-inflammatory agent into the lymphatic system and stimulation/increase in lymphatic flow pumping rate. This in turn is believed to facilitate flow of therapeutically effective amounts of the anti-inflammatory agent to therapeutic target(s), tissues, immune cells, or regions of disease or injury, in order to provide therapeutic benefit.

Methods and procedures for some of these experiments have been adapted from Aldrich, et al., Arthritis Res. Ther., (2017), 19:116 (DOI 10.1186/s13075-017-1323-z; Open Access) which is incorporated by reference herein in its entirety for all purposes.

This written description uses examples to disclose the subject matter herein, including the best mode, and also to enable any person skilled in the art to practice the subject matter this disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1.-2. (canceled)

3. A method of decreasing an elevated lymphatic amount or lymphatic concentration of an inflammatory substance in a subject having, or suspected of having, an arthritic disease or associated condition, or a symptom associated with an arthritic disease or associated condition, wherein the elevated lymphatic amount or lymphatic concentration results from the presence of the disease or associated condition, the method comprising:
placing a first medical device comprising a plurality of microneedles on the skin of the subject at a first location proximate to a first position under the skin of the subject, wherein the first position is proximate to lymph vessels and/or lymph capillaries that drain into a lymphatic duct, and wherein the microneedles of the first medical device have a surface comprising nanotopography;
inserting the plurality of microneedles of the first medical device into the subject to a depth whereby at least the epidermis is penetrated and an end of at least one of the microneedles is proximate to the first position; and
administering via the microneedles of the first medical device a therapeutically effective amount of a therapeutic agent into the first position;
wherein the therapeutically effective amount comprises an amount that results in a reduction in the lymphatic amount or lymphatic concentration of the inflammatory substance in the lymphatic system of the subject.

4. A method of increasing lymphatic pumping rate of at least on lymph node in a subject having, or suspected of having, an arthritic disease or associated condition, or a symptom associated with an arthritic disease or associated condition, the method comprising:
placing a first medical device comprising a plurality of microneedles on the skin of the subject at a first location proximate to a first position under the skin of the subject, wherein the first position is proximate to lymph vessels and/or lymph capillaries that drain into a lymphatic duct, and wherein the microneedles of the first medical device have a surface comprising nanotopography;
inserting the plurality of microneedles of the first medical device into the subject to a depth whereby at least the epidermis is penetrated and an end of at least one of the microneedles is proximate to the first position; and
administering via the microneedles of the first medical device a therapeutically effective amount of a therapeutic agent into the first position;
wherein the therapeutically effective amount comprises an amount that results in an increased lymphatic pumping rate of the at least on lymph node in the lymphatic system of the subject.

5. A method of achieving or restoring a normal pumping rate of at least one lymph node in a subject having, or suspected of having, an arthritic disease or associated condition, or a symptom associated with an arthritic disease or associated condition, wherein the pumping rate is reduced as a result of the arthritic disease or associated condition, the method comprising:
placing a first medical device comprising a plurality of microneedles on the skin of the subject at a first location proximate to a first position under the skin of the subject, wherein the first position is proximate to lymph vessels and/or lymph capillaries that drain into a lymphatic duct, and wherein the microneedles of the first medical device have a surface comprising nanotopography;
inserting the plurality of microneedles of the first medical device into the subject to a depth whereby at least the epidermis is penetrated and an end of at least one of the microneedles is proximate to the first position; and
administering via the microneedles of the first medical device a therapeutically effective amount of a therapeutic agent into the first position;
wherein the therapeutically effective amount comprises an amount that results in restoration of the normal pumping rate to a rate that is comparable to or greater than the pumping rate in a subject that does not have the disease or associated condition.

6. The method of claim 4, wherein the therapeutically effective amount comprises: an amount or concentration that is effective to treat the disease or associated condition; or an amount or concentration that is effective to reduce or eliminate at least one symptom or clinical manifestation of the disease or associated condition.

7. The method of claim 4, wherein the arthritic disease or associated condition is selected from the group consisting of rheumatoid arthritis (RA); juvenile arthritis; psoriatic arthritis; ankylosing spondylitis; gout; and combinations thereof.

8. The method of claim 4, wherein the associated condition comprises another autoimmune condition.

9. The method of claim 4, wherein the associated condition comprises an autoimmune condition selected from the group consisting of scleroderma, lupus ulcerative colitis (UC), Crohn's disease, plaque psoriasis, autoimmune uveitis, Behçet's disease, and sarcoidosis.

10. The method of claim 4, wherein the therapeutic agent comprises an anti-inflammatory agent.

11. The method of claim 4, wherein the therapeutic agent comprises an anti-arthritic agent.

12. The method of claim 4, wherein the therapeutic agent comprises an agent that reduces TNFa activity.

13. The method of claim 4, wherein the therapeutic agent is selected from the group consisting of: Adalimumab (Humira®); Adalimumab-atto (Amjevita®); Certolizumab pegol (Cimzia®); etanercept (Enbrel®); etanercept-szzs (Ereizi®); Golimumab (Simponi®, Simponi Aria®); Infliximab (Remicade®); Infliximab-dyyb (Inflectra®); analogs thereof; variants thereof; biosimilars thereof; bioequivalents thereof; and combinations thereof.

14. The method of claim 4, wherein the therapeutically effective amount of the therapeutic agent comprises a dose-sparing amount of the therapeutic agent.

15. The method of claim 4, wherein the therapeutically effective amount of the therapeutic agent is effective to reduce a DAS28 (ESR) and/or a DAS28(CRT) score by at least 10%, at least 20%, at least 30%, at least 40%, at least 50%, at least 60%, at least 70%, or greater compared to a DAS28 (ESR) and/or a DAS28(CRT) determined in the subject prior to administering the therapeutic agent.

16. The method of claim 4, wherein the therapeutically effective amount of the therapeutic agent is effective to reduce a 66/68-joint count and/or a 28-joint count score by at least 10%, at least 20%, at least 30%, at least 40%, at least 50%, at least 60%, at least 70%, or greater compared to a 66/68-joint count and/or a 28-joint count determined in the subject prior to administering the therapeutic agent.

17. The method of claim 4, wherein the therapeutically effective amount of the therapeutic agent is effective to reduce patient rating of overall disease activity by at least 10%, at least 20%, at least 30%, at least 40%, at least 50%, at least 60%, at least 70%, or greater compared to a patient rating of overall disease activity determined in the subject prior to administering the therapeutic agent.

18. The method of claim 4, wherein the therapeutically effective amount of the therapeutic agent is effective to improve ACR response by at least 10%, at least 20%, at least 30%, at least 40%, at least 50%, at least 60%, at least 70%, or greater compared to a an ACR response determined in the subject prior to administering the therapeutic agent.

19. The method of claim 4, wherein the subject is a mammal.

20. The method of claim 4, wherein the subject is a human.

21. (canceled)

22. The method of claim 4, wherein the medical device comprises a fluid delivery apparatus, wherein the fluid delivery apparatus comprises:
a fluid distribution assembly wherein a cap assembly is coupled to a cartridge assembly, and the cartridge assembly is slidably coupled to a plenum assembly, and a mechanical controller assembly is slidably coupled to the cartridge assembly;
a collet assembly constituting the housing of the fluid delivery apparatus and being slidably coupled to the fluid distribution assembly; and
a plurality of microneedles fluidically connected with the fluid distribution assembly having a surface comprising nanotopography, the plurality of microneedles being capable of penetrating the stratum corneum of the skin of a subject and controllably delivering the therapeutic agent to a depth below the surface of the skin.

23. (canceled)

24. The method of claim 4, wherein each of the microneedles in the medical device has a length between about 200 to about 800 μm, between about 250 to about 750 μm, or between about 300 to about 600 μm.

25.-26. (canceled)
